SEQUENCE LISTING (158 sequences)

SEQ ID NO: 1 - protein, 1244 aa, synthetic construct
  REGION 1..1244: note = The sequence is synthetized.
MKKIDSFTGKYSLSKTLRFRLIPVGKTLENFKNKKLLDEDMTRAENYEKVKLYMDRKHKAFIDRILQAVKLENLSEYIELYYIKNKTDKQKKDIDVFENAFRKQIAAAFKNDEERKLLFGEKMIKEVLPSFLEDEEEIKIVNTFNRFTTYFTGFYENRKNMYSADSKSSSIAYRCIDENLPRFLDNAETFRKIAISLPKENFIELTEKLSLADGFIEREFLPESFNFVLSQSGIDEYNLLLGGKTLTENGEKVKGLNEYINLFNQQHNKEEKLPHFKPLRKQILSDRQSESFIPEAFSSDNELITSIKIFFEGESSDSGYRKTIENTKRLFYNLESYGLSEIFISAGADLSEFSARAAKSWSAVRDAWIKKYDRVHSDKDKKQKKYKENRDKKYKTIKSFSLSEIQSYIRNTEFDGELPYADFDIVLFIKREISELVETSMQYYENAKQLLNTPYASEKNLLKDSNSIELIKNLLDSLKEIELYTKAFKGSGKEDEKDESFYGEYTLLSDELIVLDSLYNKVRNYLTRKPFSSDKLKLNFRNAQLLDGWDISKEKDYKTILLRKEGSYYLGIVDEEENYEKLMIDLPRPNIGDQVFEKMNYKLLPNPYKMLPKVFFSNKNKEYYSPSDEILRIRDTGSFKNGKESTTFNHGDLMKFIDFYKESIKKNNDWNIFGFKFRPTVEYKNINEFYKDVSDQGYSVSFSPVSADFIRELVEDGRLYLFRLYNKDFSENSKGIPNLHTMYFRALFDEHNLSDAVYKLNGKAEMFYRDGKLCLKDTTIHTANQSIKNKNPLQKKATSTFAYDIIKDRRYTEPQFFLHLPITMNLKAEERDSLNYDVRKALRLSEKNYIIGIDRGERNLLYISVIDQDGRIVEQESMNILSVVKDDVVYETDYHDLLDRREKERDAARKSWKSIKNIKELKEGYLSGVVNRICNLVVKYDAIIAMEDLNMGFKRSRSKVEKQVYQKFEKMLIDKMNYLVDKKADINSPGGLMKAYQLTEKFTSFKNIKFQNGFIFYVPAWLTSKIDPTTGFVSLFRTHGLSEDMCREFVSAFDLITYNNAENQFEFVVNYDNFIGSEVSPKKLWTVCTNGERVETKRSKAINNNWESKEIILTDEFVKLFKEYEIDYSDGKDIKAQCLKIEKGGFLKEVVRLFALTVQMRNSIPNTDVDYLISPVRGTDGKFFDSRKGIESLPKDADANGAYNIARKALWIINRFKESSDDELSTVKICMTQRQWLEFAQSNG

SEQ ID NO: 2 - DNA, 3735 bp, synthetic construct
  misc_feature 1..3735: note = The sequence is synthetized.
  source 1..3735: mol_type = other DNA
atgaaaaagattgattcgttcacaggtaaatattctctctcaaaaaccttgcgttttcggctgattccggtaggaaaaacgcttgagaatttcaaaaacaaaaagttgcttgatgaagatatgaccagagccgagaactatgaaaaggtcaaattatatatggacagaaaacataaagcgtttatagacagaatattacaagcggtaaaacttgaaaatctctctgaatatatcgaattgtattatataaagaataaaacggataaacaaaaaaaagacattgacgtttttgaaaatgctttcagaaaacagattgccgctgctttcaagaatgacgaggaaagaaaacttcttttcggagaaaaaatgattaaagaggttcttccctcatttcttgaagacgaggaagagattaaaattgtaaacacgtttaacagatttacaacatattttacggggttttacgaaaaccgtaagaatatgtattcagccgactcaaaatcaagttcaatcgcatatcgttgtatagatgaaaatcttccgcgatttttggataacgcagagacttttcgtaaaattgcaatatctttgccgaaagagaactttatagaattgactgaaaaactatctctcgcagatgggtttattgagagggaatttcttcctgaaagttttaattttgtcctttctcagtctggtattgatgaatacaacttgcttctcggaggaaaaacattgacggaaaacggcgaaaaagtgaaaggtcttaatgaatatatcaatctttttaatcagcaacacaacaaagaagaaaagcttccccatttcaaacctctgagaaaacaaattctcagtgacagacagtcagaatcttttataccggaagctttcagctcggacaatgaattgataacttcaatcaaaatattttttgaaggggaatcaagtgattccggatatagaaagacaatagaaaataccaagagattattttataatcttgagagttatggactgtccgaaatttttatttcggcgggtgcagacctttcagagttttctgctcgtgccgctaaaagttggagcgctgtacgtgatgcatggataaaaaagtatgacagggtacattcagataaagacaaaaaacaaaaaaaatacaaggaaaaccgtgataagaaatataagacaattaaaagcttttcattgtctgagatacaatcgtatataaggaatacggagtttgatggtgaattgccgtatgctgattttgatattgttctgtttattaaacgtgaaatttcggaattggtcgagacttcaatgcaatattacgagaatgcaaaacaacttttgaatactccttatgcaagtgagaaaaatttgcttaaagacagtaacagtattgagttgataaagaatcttcttgacagcctgaaagaaattgaattatatacaaaagcattcaaaggcagtggtaaagaagatgaaaaagatgagagtttctatggtgaatacacgttgctttcggatgagctgattgtacttgactcgctttataataaagtaagaaattatctgacaagaaaacctttttcctcggataaattgaaattgaattttcgtaacgcacagttgttggacggctgggatatcagcaaagaaaaagattataaaacaattttactgcgaaaagaaggctcctattatctcggaatcgtagatgaagaagaaaactatgaaaaactcatgattgatttgccgagaccgaatatcggagatcaggtttttgaaaagatgaattataagcttctaccgaatccttataaaatgttgccgaaagtgtttttcagcaacaaaaataaggaatattattctccgagcgatgagatattgcggatacgggatacgggaagtttcaaaaacggaaaagaatcgacaacttttaatcatggcgaccttatgaagtttatcgatttttataaagagtcaatcaagaaaaacaacgattggaatatcttcggtttcaaatttagacctacggtcgaatataaaaatattaatgagttttacaaagatgtttcggatcaaggatattccgtttctttctctcctgtgtcggcagattttatcagagaacttgttgaagatggaagactttatcttttcagactttataataaagacttttccgagaactccaaggggattccgaatttgcatactatgtacttccgtgcattatttgatgagcacaatctttccgatgctgtatacaagctgaacggcaaagccgaaatgttttacagagacggtaaactttgcttaaaagataccactatacatacagcaaatcaatcgattaaaaataagaatcctttgcagaaaaaagcgaccagtacttttgcatatgatataatcaaagacagaaggtataccgaacctcaatttttcttgcatcttcctataaccatgaacttaaaagcagaggagagagattctcttaattacgacgtgcgaaaggcgctgagactttccgaaaagaactatattataggcattgatcgcggcgaaagaaatcttttgtatataagcgttatagatcaagatggtcgtattgttgaacaagagtcgatgaatattctttcagttgtgaaagatgatgttgtttatgaaacagattatcatgatcttcttgatcgcagggagaaggaaagggatgcagcgagaaaaagttggaaatcaattaaaaacattaaggaattgaaagagggctatctcagtggtgttgttaacagaatttgtaatcttgtagttaagtatgatgccattattgctatggaagaccttaatatgggctttaagcgcagccgttcaaaggttgaaaaacaggtttatcagaaatttgaaaaaatgctgattgacaaaatgaactatttggttgacaaaaaagccgatattaattctcccggcggcttgatgaaagcatatcagcttacagaaaaatttacgagtttcaaaaatataaagtttcaaaacggttttattttctatgttcccgcatggttgaccagcaaaatagatcccacgaccggttttgtatctttgttcagaacacacggactatcggaagatatgtgccgggagtttgtttccgcatttgatttaattacatacaacaatgcggaaaatcaatttgaatttgttgtaaattacgataattttatagggtcggaagtttctccgaaaaaattatggacagtttgcactaacggtgaaagggttgaaacaaaaagaagcaaagccataaataataattgggagtcaaaagaaattatactgacggatgagtttgttaaattgtttaaagaatatgaaatagactattcagacggaaaagatataaaagcgcaatgcttaaaaatcgaaaaaggaggatttctcaaagaagttgtcagactgtttgcattgactgttcaaatgcgaaacagtattccgaatacagatgttgattatctgatttctcctgtaagaggaaccgacggcaagttcttcgacagccgtaaaggcatagaatccctgccgaaagatgcagacgcaaacggtgcatataatatcgcacgtaaagcattgtggataataaacagatttaaggaaagttctgatgatgagttatcaacggtaaaaatctgcatgacgcaacggcaatggcttgaatttgctcaaagcaatggctga

SEQ ID NO: 3 - DNA, 3783 bp, synthetic construct
  misc_feature 1..3783: note = The sequence is synthetized.
  source 1..3783: mol_type = other DNA
atgggcaaaaagatagattcatttacaggaaaatactccctgtcgaagacgctgcgctttcgtttgattccagttggtaagacgcttgagaacttcaaaaataaaaaattgctagacgaagacatgacccgtgccgagaactacgaaaaggtgaagttgtacatggatcgcaagcacaaagccttcatagaccgcatcctgcaggcggtcaagctcgagaatctgagcgaatatatcgagttgtactacatcaagaataagaccgataagcaaaaaaaagacattgacgtttttgaaaacgcctttagaaaacaaattgcggccgcgttcaaaaacgatgaagagaggaagctccttttcggtgaaaagatgattaaggaagtgctgccgagcttcttggaggacgaagaagaaatcaaaatcgtgaacacttttaaccgcttcaccacatacttcaccggcttttacgagaaccgcaagaatatgtatagcgcggactctaaatccagcagcatcgcgtatcgctgcattgatgagaacctgcctcgctttctggacaacgccgaaaccttccggaagattgcgatttctctgccgaaagagaatttcattgaactgactgagaaattgtccctggcagatggttttatcgaacgggagtttctgccggaatctttcaacttcgtgttgtcacagtcaggtattgatgagtacaatctactgctgggtggtaaaaccctgaccgaaaacggcgagaaagtcaaaggcctgaacgagtacataaacctgttcaaccagcagcataacaaagaagagaagttgccgcactttaagccgctgcgtaaacaaatcctgagcgatcgtcagagcgaatcttttatcccggaagcattcagcagcgataacgaattgattacctcgatcaagatttttttcgaaggcgagtcgtctgacagcggttataggaaaaccattgagaatacgaaacgcttattctataatcttgagagttacggcctgtctgaaatctttatctcggctggtgcggacctaagcgagttcagcgcgcgtgcggctaagagctggagcgcggttcgtgatgcatggattaaaaagtacgaccgcgtccatagcgacaaggacaaaaagcagaaaaaatacaaagagaaccgtgataaaaaatacaaaaccatcaagagcttctctctgtccgagattcagtcttatatccgtaacacggagttcgacggtgaattaccgtacgcagactttgacatcgttctgttcatcaagcgtgaaatcagcgaactggttgaaacgagcatgcagtattatgaaaacgcgaaacaactgctgaacacaccgtatgctagcgaaaaaaacctgcttaaggacagcaattcaattgagctgatcaagaatttgctcgactccctgaaagagatagagctgtacaccaaggcgttcaagggtagcggaaaggaagatgagaaggacgaatcgttctacggtgaatataccttgctctccgacgagctgattgttctggatagcctgtacaacaaggtacgtaattatttgacccgtaaaccgtttagttcggacaaactgaagctgaacttccgcaacgcccaactgctggacggctgggatattagcaaggagaaggattataaaaccattttgctgcgtaaggagggttcctattacctgggtatcgtcgatgaagaagagaactatgaaaagctgatgatcgatctgccgcgtcctaatattggcgaccaagtttttgagaaaatgaactacaagctgctgccaaacccgtacaagatgctgccgaaggtgttcttcagcaacaagaacaaagaatactacagcccgagcgacgagattctgcgtattcgtgacaccggtagctttaagaacggcaaggagagcacgacttttaatcatggcgatctgatgaaatttatcgatttttataaagaatctatcaagaaaaacaatgattggaatatcttcggctttaaattccgtccgaccgttgaatacaagaacatcaacgaattctacaaagacgtctccgaccaaggttattccgtgagctttagcccggttagcgctgactttatccgtgaactggtcgaagatggccgtctgtacttatttcgcctatacaataaggacttcagcgagaactccaaaggcattccgaatctacacactatgtattttagagcgctctttgacgagcacaatctgtcagacgctgtgtacaaattgaatggcaaggctgaaatgttttaccgtgacggtaaattatgcctgaaagacactacgatccacaccgcaaatcaatccattaagaataagaatccgttgcaaaagaaagcgaccagcacgtttgcgtatgatatcattaaagaccgtcgttataccgaaccgcagttttttctgcacctgccgattaccatgaatcttaaggcggaggagcgtgattcgttgaattacgacgttagaaaagcgctgcgtctgtctgagaagaactatattattggtatcgatcgcggtgaacgtaacttgttgtacatctctgttattgatcaggatggccgtatcgtggaacaggagtctatgaacatcctgtccgtggtgaaagacgatgtggtttacgaaaccgactaccatgatctgcttgaccgtcgtgagaaggagcgcgacgcggcgcgcaaaagttggaaatccatcaaaaacatcaaagaattaaaggaaggctatctgagcggagttgtgaaccgtatttgcaacctggtagttaagtacgacgccatcattgctatggaggatctaaacatgggtttcaaacgcagccgtagcaaagtggaaaagcaagtgtatcagaagtttgaaaagatgttgattgacaagatgaattacctggtagataagaaagccgatattaactccccgggtggcttgatgaaggcgtaccaattgaccgagaaattcaccagcttcaagaacatcaagttccagaatggcttcatcttctacgtgccggcatggctgactagcaaaatcgatccgacgaccggttttgtttccttgttccgcacccacggtctgtccgaagatatgtgtcgtgagtttgtgagcgcctttgatctgatcacctataacaacgccgaaaatcagtttgaattcgtcgttaattatgacaactttatcggcagtgaggttagcccgaaaaagctgtggaccgtctgtaccaatggtgagcgtgttgagaccaaacgtagcaaggcaattaataacaactgggaatccaaagagatcatccttaccgacgagtttgttaagttgttcaaagaatacgagatcgactacagcgacggcaaggacatcaaagctcaatgcctcaaaatcgaaaaaggcggtttcttgaaagaggttgtgcgcctgtttgcgctgaccgttcagatgcgcaacagcatcccgaataccgatgtggactatctgatttctcccgtgcgcggaaccgacggtaaattcttcgattcccgtaagggcatcgaaagcctgccaaaggatgccgacgcgaatggcgcgtacaacattgcgcgtaaggcgctgtggattattaaccgtttcaaggagagctccgatgatgagttaagcacggtgaagatctgcatgacccagcgtcagtggctggagttcgcacaaagcaacggcaaaaggccggcggccacgaaaaaggccggccaggcaaaaaagaaaaag

SEQ ID NO: 4 - DNA, 3834 bp, synthetic construct
  misc_feature 1..3834: note = The sequence is synthetized.
  source 1..3834: mol_type = other DNA
gccaccatggccccaaagaagaagcggaaggtcggtatccacggagtcccagcagccaagaagatcgatagcttcaccggcaaatatagcctctcaaaaaccctgcgcttccggctgatacctgtgggcaagaccctggagaacttcaaaaacaagaagctgctggacgaggatatgacaagagccgagaactacgagaaggtcaagctgtacatggacagaaagcacaaggcctttattgacagaatcctgcaggccgtgaagctcgagaatctgagcgagtacatcgagctgtactacattaagaataaaaccgacaagcagaagaaggacatcgatgtgttcgagaacgccttcagaaagcagatcgctgccgccttcaagaacgacgaggaaagaaagctgctgttcggcgagaagatgatcaaggaagtgctgcccagcttcctggaagatgaagaagagatcaaaattgtgaacacctttaacagattcaccacatactttacaggcttttacgagaatagaaagaatatgtactctgctgacagcaagagcagcagcatcgcctatcggtgcatcgacgaaaacctgcctcggttcctggacaacgccgagaccttcagaaaaatcgccatcagcctgcctaaggaaaacttcatcgagctgaccgagaagctgtctctcgccgacggcttcatcgagagagagttcctgccagagagtttcaacttcgtgctgagccagtctgggatcgacgagtacaacctgctgctgggaggcaagaccctgaccgagaacggcgaaaaagtgaaaggcctgaacgagtatatcaacctgtttaaccagcagcacaacaaggaagagaagttgccccacttcaagcctctcagaaagcagatcctgtccgacaggcagtctgagtcttttatccctgaggccttctcctccgacaacgagctgatcacaagcatcaagatcttcttcgaaggcgagtcatcggatagcggataccggaagaccatcgagaacaccaagagactgttctataacctggaatcttacggcctgagcgagatcttcatatctgccggcgccgacctgagcgaattttctgccagagccgccaagtcttggtccgccgtgcgggacgcctggatcaaaaagtacgacagagtgcacagcgataaggacaaaaagcaaaagaaatacaaggagaacagagataagaaatataaaaccatcaagtcttttagcttatctgaaatccagagctacattcggaatactgagttcgatggcgagctgccctacgccgactttgacatcgtgctgttcatcaaaagggagattagcgagctcgtggagaccagcatgcaatactacgagaacgccaaacagctgctgaacacaccttacgccagcgagaaaaatctgctgaaggattctaattctatcgagctgatcaagaacctgcttgatagcctgaaagagatcgagctgtatacaaaggccttcaaaggcagcggtaaggaggatgagaaagacgagtctttctacggcgagtacacactgctgagcgacgagctcattgtgctggatagcctatacaacaaggtgagaaactacctcacaagaaaacccttcagctccgataagctgaagctgaatttccggaatgcccagctgcttgatggctgggacattagcaaggaaaaggactacaaaaccatcctgctaagaaaggaaggatcttactacctgggcatcgtggacgaggaagaaaactacgagaagctgatgatcgatctgcctagacccaacatcggagatcaagtgtttgagaagatgaactacaagctgctgcctaacccttacaagatgctgcctaaggtgttcttcagcaacaagaacaaggagtattatagccctagcgacgaaatcctgagaatccgggatacaggctctttcaagaatggaaaggagagcaccacattcaaccacggcgacctgatgaagttcatcgacttctacaaggaaagcatcaagaagaataacgactggaacatcttcggctttaagttcagacctacagtggagtacaagaacatcaatgagttctacaaggacgtgagcgaccagggctacagtgtgtcattcagccccgtgtccgccgatttcatccgtgaactggtggaagacggcagactctacctgttcagactgtacaataaggactttagcgagaatagcaagggcatccccaacctgcacaccatgtacttcagagccctgtttgatgagcacaacctgagcgacgccgtgtataaactgaatggcaaggccgagatgttctacagagacggcaagctgtgcctgaaggacacaaccatccacacagccaaccagtctatcaagaacaaaaaccccctgcaaaagaaggccaccagcacatttgcctacgacatcatcaaggaccgtcggtacaccgaacctcagttcttcctgcatctgccaatcaccatgaatctcaaggccgaggaacgggacagcctgaactacgacgtgcggaaggctctgagactgtccgagaaaaactacatcattggaatcgaccggggagagagaaacctgctttacatcagcgtgatcgaccaggacggcagaatcgtcgagcaggagtccatgaacattctgtctgtcgtgaaagacgatgtggtgtacgaaaccgactaccacgatctactggatagaagagagaaggagagagacgccgctagaaagtcctggaagagcatcaagaacatcaaggaactgaaggaaggctacctgagcggcgtggttaatcggatctgcaacctggtcgtgaagtacgacgctatcatcgccatggaagatctgaacatgggcttcaagcggagcagaagcaaggttgagaaacaggtgtaccagaaattcgagaagatgctgatcgataagatgaattatctggtcgacaagaaggccgacatcaacagccctggcggcctgatgaaggcttaccagctgaccgagaagtttaccagctttaaaaacatcaaattccagaacggctttatcttctacgttcctgcttggctgaccagcaagatcgatcctaccaccggcttcgtgagcctgttccggacccacggcctgtccgaagacatgtgcagagagttcgtgtctgctttcgacctgatcacctacaacaatgccgaaaatcagttcgaatttgtggtgaactacgataacttcattggatcagaggtgtctccaaagaaactgtggaccgtgtgcaccaacggcgaaagagtggaaacaaagcgcagcaaggcaatcaacaacaactgggaatccaaggaaatcatcctgaccgacgagttcgtgaagctgttcaaggagtacgagatcgactactccgacgggaaagacatcaaagctcaatgtctgaagatcgagaagggcggattcctgaaggaggtggtgaggctgttcgccctgacagtccagatgagaaacagcatccctaacaccgatgtggattacctgatcagccccgtgcggggcaccgatggaaaatttttcgacagccggaaaggcatcgagagcctgcctaaggatgccgacgctaatggagcctacaacatcgctagaaaggccctgtggatcatcaacagattcaaagaaagcagcgacgacgagctgtccacagtgaagatctgtatgacccagagacagtggctggagttcgcccagagcaacggtaaaaggccggcggccacgaaaaaggccggccaggcaaaaaagaaaaag

SEQ ID NO: 5 - DNA, 752 bp, African swine fever virus
  source 1..752: mol_type = other DNA
ctgtaacgcagcacagctgaaccgttctgaagaagaagaaagttaatagcagatgccgataccacaagatcagccgtagtgatagaccccacgtaatccgtgtcccaactaatataaaattctcttgctctggatacgttaatatgaccactgggttggtattcctcccgtggcttcaaagcaaaggtaatcatcatcgcacccggatcatcgggggttttaatcgcattgcctccgtagtggaagggtatgtaagagctgcagaactttgatggaaatttatcgataagattgataccatgagcagttacggaaatgtttttaataataggtaatgtgatcggatacgtaacggggctaatatcagatatagatgaacatgcgtctggaagagctgtatctctatcctgaaagcttatctctgcgtggtgagtgggctgcataatggcgttaacaacatgtccgaacttgtgccaatctcggtgttgatgaggattttgatcggagatgttccaggtaggttttaatcctataaacatatattcaatgggccatttaagagcagacattagtttttcatcgtggtggttattgttggtgtgggtcacctgcgttttatggacacgtatcagcgaaaagcgaacgcgttttacaaaaaggttgtgtatttcaggggttacaaacaggttattgatgtaaagttcattattcgtgagcgagatttcattaatgactcctgggataaaccatgg

SEQ ID NO: 6 - DNA, 97 bp, synthetic construct
  misc_feature 1..97: note = The sequence is synthetized
  misc_difference 30..36: note = n is a, c, g, or t
  source 1..97: mol_type = other DNA
ggccagtgaattcgagctcggtacccgggnnnnnnngagaagtcatttaataaggccactgttaaaaagcttggcgtaatcatggtcatagctgttt

SEQ ID NO: 7 - DNA, 21 bp, synthetic construct
  misc_feature 1..21: note = The sequence is synthetized.
  source 1..21: mol_type = other DNA
ggccagtgaattcgagctcgg

SEQ ID NO: 8 - DNA, 27 bp, synthetic construct
  misc_feature 1..27: note = The sequence is synthetized.
  source 1..27: mol_type = other DNA
aaacagctatgaccatgattacgccaa

SEQ ID NO: 9 - RNA, 44 nt, synthetic construct
  misc_feature 1..44: note = The sequence is synthetized.
  source 1..44: mol_type = other RNA
aatttctactattgtagattgagaagtcatttaataaggccact

SEQ ID NO: 10 - DNA, 28 bp, synthetic construct
  misc_feature 1..28: note = The sequence is synthetized.
  source 1..28: mol_type = other DNA
caatttcacacaggaaacagctatgacc

SEQ ID NO: 11 - DNA, 20 bp, synthetic construct
  misc_feature 1..20: note = The sequence is synthetized.
  source 1..20: mol_type = other DNA
ctgtaacgcagcacagctga

SEQ ID NO: 12 - DNA, 18 bp, synthetic construct
  misc_feature 1..18: note = The sequence is synthetized.
  source 1..18: mol_type = other DNA
catatattcaatgggcca

SEQ ID NO: 13 - DNA, 17 bp, synthetic construct
  misc_feature 1..17: note = The sequence is synthetized.
  source 1..17: mol_type = other DNA
atcgtggtggttattgt

SEQ ID NO: 14 - DNA, 20 bp, synthetic construct
  misc_feature 1..20: note = The sequence is synthetized.
  source 1..20: mol_type = other DNA
ccatggtttatcccaggagt

SEQ ID NO: 15 - DNA, 18 bp, synthetic construct
  misc_feature 1..18: note = The sequence is synthetized.
  source 1..18: mol_type = other DNA
tttcgctgatacgtgtcc

SEQ ID NO: 16 - DNA, 59 bp, synthetic construct
  misc_feature 1..59: note = The sequence is synthetized.
  source 1..59: mol_type = other DNA
catatattcaatgggccaaataagagcagacattagtttttcatcgtggtggttattgt

SEQ ID NO: 17 - DNA, 59 bp, synthetic construct
  misc_feature 1..59: note = The sequence is synthetized.
  source 1..59: mol_type = other DNA
catatattcaatgggccaaattagagcagacattagtttttcatcgtggtggttattgt

SEQ ID NO: 18 - DNA, 59 bp, synthetic construct
  misc_feature 1..59: note = The sequence is synthetized.
  source 1..59: mol_type = other DNA
catatattcaatgggccaaatgagagcagacattagtttttcatcgtggtggttattgt

SEQ ID NO: 19 - DNA, 59 bp, synthetic construct
  misc_feature 1..59: note = The sequence is synthetized.
  source 1..59: mol_type = other DNA
catatattcaatgggccaaatcagagcagacattagtttttcatcgtggtggttattgt

SEQ ID NO: 20 - DNA, 59 bp, synthetic construct
  misc_feature 1..59: note = The sequence is synthetized.
  source 1..59: mol_type = other DNA
catatattcaatgggccaagtaagagcagacattagtttttcatcgtggtggttattgt

SEQ ID NO: 21 - DNA, 59 bp, synthetic construct
  misc_feature 1..59: note = The sequence is synthetized.
  source 1..59: mol_type = other DNA
catatattcaatgggccaagttagagcagacattagtttttcatcgtggtggttattgt

SEQ ID NO: 22 - DNA, 59 bp, synthetic construct
  misc_feature 1..59: note = The sequence is synthetized.
  source 1..59: mol_type = other DNA
catatattcaatgggccaagtgagagcagacattagtttttcatcgtggtggttattgt

SEQ ID NO: 23 - DNA, 59 bp, synthetic construct
  misc_feature 1..59: note = The sequence is synthetized.
  source 1..59: mol_type = other DNA
catatattcaatgggccaagtcagagcagacattagtttttcatcgtggtggttattgt

SEQ ID NO: 24 - DNA, 59 bp, synthetic construct
  misc_feature 1..59: note = The sequence is synthetized.
  source 1..59: mol_type = other DNA
catatattcaatgggccaactaagagcagacattagtttttcatcgtggtggttattgt

SEQ ID NO: 25 - DNA, 59 bp, synthetic construct
  misc_feature 1..59: note = The sequence is synthetized.
  source 1..59: mol_type = other DNA
catatattcaatgggccaacttagagcagacattagtttttcatcgtggtggttattgt

SEQ ID NO: 26 - DNA, 59 bp, synthetic construct
  misc_feature 1..59: note = The sequence is synthetized.
  source 1..59: mol_type = other DNA
catatattcaatgggccaactgagagcagacattagtttttcatcgtggtggttattgt

SEQ ID NO: 27 - DNA, 59 bp, synthetic construct
  misc_feature 1..59: note = The sequence is synthetized.
  source 1..59: mol_type = other DNA
catatattcaatgggccaactcagagcagacattagtttttcatcgtggtggttattgt

SEQ ID NO: 28 - DNA, 59 bp, synthetic construct
  misc_feature 1..59: note = The sequence is synthetized.
  source 1..59: mol_type = other DNA
catatattcaatgggccaattaagagcagacattagtttttcatcgtggtggttattgt

SEQ ID NO: 29 - DNA, 59 bp, synthetic construct
  misc_feature 1..59: note = The sequence is synthetized.
  source 1..59: mol_type = other DNA
catatattcaatgggccaatttagagcagacattagtttttcatcgtggtggttattgt

SEQ ID NO: 30 - DNA, 59 bp, synthetic construct
  misc_feature 1..59: note = The sequence is synthetized.
  source 1..59: mol_type = other DNA
catatattcaatgggccaattgagagcagacattagtttttcatcgtggtggttattgt

SEQ ID NO: 31 - DNA, 59 bp, synthetic construct
  misc_feature 1..59: note = The sequence is synthetized.
  source 1..59: mol_type = other DNA
catatattcaatgggccaattcagagcagacattagtttttcatcgtggtggttattgt

SEQ ID NO: 32 - DNA, 59 bp, synthetic construct
  misc_feature 1..59: note = The sequence is synthetized.
  source 1..59: mol_type = other DNA
catatattcaatgggccacccaagagcagacattagtttttcatcgtggtggttattgt

SEQ ID NO: 33 - DNA, 59 bp, synthetic construct
  misc_feature 1..59: note = The sequence is synthetized.
  source 1..59: mol_type = other DNA
catatattcaatgggccatttaagagcagacattagtttttcatcgtggtggttattgt

SEQ ID NO: 34 - DNA, 59 bp, synthetic construct
  misc_feature 1..59: note = The sequence is synthetized.
  source 1..59: mol_type = other DNA
catatattcaatgggccacgccagagcagacattagtttttcatcgtggtggttattgt

SEQ ID NO: 35 - DNA, 59 bp, synthetic construct
  misc_feature 1..59: note = The sequence is synthetized.
  source 1..59: mol_type = other DNA
catatattcaatgggccacggcagagcagacattagtttttcatcgtggtggttattgt

SEQ ID NO: 36 - DNA, 59 bp, synthetic construct
  misc_feature 1..59: note = The sequence is synthetized.
  source 1..59: mol_type = other DNA
catatattcaatgggccaccgcagagcagacattagtttttcatcgtggtggttattgt

SEQ ID NO: 37 - DNA, 59 bp, synthetic construct
  misc_feature 1..59: note = The sequence is synthetized.
  source 1..59: mol_type = other DNA
catatattcaatgggccagcccagagcagacattagtttttcatcgtggtggttattgt

SEQ ID NO: 38 - DNA, 59 bp, synthetic construct
  misc_feature 1..59: note = The sequence is synthetized.
  source 1..59: mol_type = other DNA
catatattcaatgggccagcgcagagcagacattagtttttcatcgtggtggttattgt

SEQ ID NO: 39 - DNA, 59 bp, synthetic construct
  misc_feature 1..59: note = The sequence is synthetized.
  source 1..59: mol_type = other DNA
catatattcaatgggccaggccagagcagacattagtttttcatcgtggtggttattgt

SEQ ID NO: 40 - DNA, 59 bp, synthetic construct
  misc_feature 1..59: note = The sequence is synthetized.
  source 1..59: mol_type = other DNA
catatattcaatgggccagggcagagcagacattagtttttcatcgtggtggttattgt

SEQ ID NO: 41 - DNA, 752 bp, synthetic construct
  misc_feature 1..752: note = The sequence is synthetized.
  misc_difference 555..558: note = n is a, c, g, or t
  source 1..752: mol_type = other DNA
ctgtaacgcagcacagctgaaccgttctgaagaagaagaaagttaatagcagatgccgataccacaagatcagccgtagtgatagaccccacgtaatccgtgtcccaactaatataaaattctcttgctctggatacgttaatatgaccactgggttggtattcctcccgtggcttcaaagcaaaggtaatcatcatcgcacccggatcatcgggggttttaatcgcattgcctccgtagtggaagggtatgtaagagctgcagaactttgatggaaatttatcgataagattgataccatgagcagttacggaaatgtttttaataataggtaatgtgatcggatacgtaacggggctaatatcagatatagatgaacatgcgtctggaagagctgtatctctatcctgaaagcttatctctgcgtggtgagtgggctgcataatggcgttaacaacatgtccgaacttgtgccaatctcggtgttgatgaggattttgatcggagatgttccaggtaggttttaatcctataaacatatattcaatgggccannnnagagcagacattagtttttcatcgtggtggttattgttggtgtgggtcacctgcgttttatggacacgtatcagcgaaaagcgaacgcgttttacaaaaaggttgtgtatttcaggggttacaaacaggttattgatgtaaagttcattattcgtgagcgagatttcattaatgactcctgggataaaccatgg

SEQ ID NO: 42 - RNA, 40 nt, synthetic construct
  misc_feature 1..40: note = The sequence is synthetized.
  source 1..40: mol_type = other RNA
aatttctactattgtagattagagcagacattagtttttc

SEQ ID NO: 43 - DNA, 40 bp, synthetic construct
  source 1..40: mol_type = other DNA
  misc_feature 1..20: note = RNA
  misc_feature 21..40: note = DNA
aatttctactattgtagattcggttatccacagaatcagg

SEQ ID NO: 44 - DNA, 40 bp, synthetic construct
  misc_feature 21..40: note = DNA
  source 1..40: mol_type = other DNA
  misc_feature 1..20: note = RNA
aatttctactattgtagattaatgtgagttagctcactca

SEQ ID NO: 45 - DNA, 40 bp, synthetic construct
  misc_feature 21..40: note = DNA
  source 1..40: mol_type = other DNA
  misc_feature 1..20: note = RNA
aatttctactattgtagatttgctctgatgccgcatagtt

SEQ ID NO: 46 - DNA, 40 bp, synthetic construct
  misc_feature 21..40: note = DNA
  source 1..40: mol_type = other DNA
  misc_feature 1..20: note = RNA
aatttctactattgtagattaatcggccaacgcgcgggga

SEQ ID NO: 47 - DNA, 40 bp, synthetic construct
  misc_feature 21..40: note = DNA
  source 1..40: mol_type = other DNA
  misc_feature 1..20: note = RNA
aatttctactattgtagattagcgggcagtgagcgcaacg

SEQ ID NO: 48 - DNA, 40 bp, synthetic construct
  misc_feature 21..40: note = DNA
  source 1..40: mol_type = other DNA
  misc_feature 1..20: note = RNA
aatttctactattgtagattcacacaggaaacagctatga

SEQ ID NO: 49 - DNA, 40 bp, synthetic construct
  misc_feature 21..40: note = DNA
  source 1..40: mol_type = other DNA
  misc_feature 1..20: note = RNA
aatttctactattgtagattattaatgcagctggcacgac

SEQ ID NO: 50 - DNA, 40 bp, synthetic construct
  misc_feature 21..40: note = DNA
  source 1..40: mol_type = other DNA
  misc_feature 1..20: note = RNA
aatttctactattgtagattggcgctcttccgcttcctcg

SEQ ID NO: 51 - DNA, 41 bp, synthetic construct
  misc_feature 21..41: note = DNA
  source 1..41: mol_type = other DNA
  misc_feature 1..20: note = RNA
aatttctactattgtagatttttctcagaatgacttggttg

SEQ ID NO: 52 - DNA, 40 bp, synthetic construct
  misc_feature 21..40: note = DNA
  source 1..40: mol_type = other DNA
  misc_feature 1..20: note = RNA
aatttctactattgtagattgagcgtcgatttttgtgatg

SEQ ID NO: 53 - DNA, 40 bp, synthetic construct
  misc_feature 21..40: note = DNA
  source 1..40: mol_type = other DNA
  misc_feature 1..20: note = RNA
aatttctactattgtagatttagaggatccccgggtaccg

SEQ ID NO: 54 - DNA, 40 bp, synthetic construct
  misc_feature 21..40: note = DNA
  source 1..40: mol_type = other DNA
  misc_feature 1..20: note = RNA
aatttctactattgtagattactcgctgcgctcggtcgtt

SEQ ID NO: 55 - DNA, 40 bp, synthetic construct
  misc_feature 21..40: note = DNA
  source 1..40: mol_type = other DNA
  misc_feature 1..20: note = RNA
aatttctactattgtagatttgagtattcaacatttccgt

SEQ ID NO: 56 - DNA, 40 bp, synthetic construct
  misc_feature 21..40: note = DNA
  source 1..40: mol_type = other DNA
  misc_feature 1..20: note = RNA
aatttctactattgtagattgggtaacgccagggttttcc

SEQ ID NO: 57 - DNA, 40 bp, synthetic construct
  misc_feature 21..40: note = DNA
  source 1..40: mol_type = other DNA
  misc_feature 1..20: note = RNA
aatttctactattgtagattagaggtggcgaaacccgaca

SEQ ID NO: 58 - DNA, 40 bp, synthetic construct
  misc_feature 21..40: note = DNA
  source 1..40: mol_type = other DNA
  misc_feature 1..20: note = RNA
aatttctactattgtagattagctgataccgctcgccgca

SEQ ID NO: 59 - RNA, 40 nt, synthetic construct
  misc_feature 1..40: note = The sequence is synthetized.
  source 1..40: mol_type = other RNA
aatttctactattgtagattataccatgagcagttacgga

SEQ ID NO: 60 - RNA, 40 nt, synthetic construct
  misc_feature 1..40: note = The sequence is synthetized.
  source 1..40: mol_type = other RNA
aatttctactattgtagattaagccacgggaggaatacca

SEQ ID NO: 61 - RNA, 40 nt, synthetic construct
  misc_feature 1..40: note = The sequence is synthetized.
  source 1..40: mol_type = other RNA
aatttctactattgtagattcactacggaggcaatgcgat

SEQ ID NO: 62 - RNA, 40 nt, synthetic construct
  misc_feature 1..40: note = The sequence is synthetized.
  source 1..40: mol_type = other RNA
aatttctactattgtagattcgtaactgctcatggtatca

SEQ ID NO: 63 - RNA, 40 nt, synthetic construct
  misc_feature 1..40: note = The sequence is synthetized.
  source 1..40: mol_type = other RNA
aatttctactattgtagattaaagcaaaggtaatcatcat

SEQ ID NO: 64 - RNA, 40 nt, synthetic construct
  misc_feature 1..40: note = The sequence is synthetized.
  source 1..40: mol_type = other RNA
aatttctactattgtagattgatggaaatttatcgataag

SEQ ID NO: 65 - RNA, 40 nt, synthetic construct
  misc_feature 1..40: note = The sequence is synthetized.
  source 1..40: mol_type = other RNA
aatttctactattgtagattcatacccttccactacggag

SEQ ID NO: 66 - RNA, 40 nt, synthetic construct
  misc_feature 1..40: note = The sequence is synthetized.
  source 1..40: mol_type = other RNA
aatttctactattgtagattcggaaatgtttttaataata

SEQ ID NO: 67 - RNA, 40 nt, synthetic construct
  misc_feature 1..40: note = The sequence is synthetized.
  source 1..40: mol_type = other RNA
aatttctactattgtagattatctatatctgatattagcc

SEQ ID NO: 68 - RNA, 40 nt, synthetic construct
  misc_feature 1..40: note = The sequence is synthetized.
  source 1..40: mol_type = other RNA
aatttctactattgtagattttaataataggtaatgtgat

SEQ ID NO: 69 - RNA, 40 nt, synthetic construct
  misc_feature 1..40: note = The sequence is synthetized.
  source 1..40: mol_type = other RNA
aatttctactattgtagattcaaaggtaatcatcatcgca

SEQ ID NO: 70 - RNA, 40 nt, synthetic construct
  misc_feature 1..40: note = The sequence is synthetized.
  source 1..40: mol_type = other RNA
aatttctactattgtagattttatcgataagattgatacc

SEQ ID NO: 71 - RNA, 40 nt, synthetic construct
  misc_feature 1..40: note = The sequence is synthetized.
  source 1..40: mol_type = other RNA
aatttctactattgtagattatatattcaatgggccattt

SEQ ID NO: 72 - RNA, 40 nt, synthetic construct
  misc_feature 1..40: note = The sequence is synthetized.
  source 1..40: mol_type = other RNA
aatttctactattgtagatttcctcatcaacaccgagatt

SEQ ID NO: 73 - RNA, 40 nt, synthetic construct
  misc_feature 1..40: note = The sequence is synthetized.
  source 1..40: mol_type = other RNA
aatttctactattgtagattacgtaatccgtgtcccaact

SEQ ID NO: 74 - RNA, 40 nt, synthetic construct
  misc_feature 1..40: note = The sequence is synthetized.
  source 1..40: mol_type = other RNA
aatttctactattgtagatttggcttcaaagcaaaggtaa

SEQ ID NO: 75 - RNA, 40 nt, synthetic construct
  misc_feature 1..40: note = The sequence is synthetized.
  source 1..40: mol_type = other RNA
aatttctactattgtagattactaatataaaattctcttg

SEQ ID NO: 76 - RNA, 40 nt, synthetic construct
  misc_feature 1..40: note = The sequence is synthetized.
  source 1..40: mol_type = other RNA
aatttctactattgtagatttccactacggaggcaatgcg

SEQ ID NO: 77 - RNA, 40 nt, synthetic construct
  misc_feature 1..40: note = The sequence is synthetized.
  source 1..40: mol_type = other RNA
aatttctactattgtagatttgcataatggcgttaacaac

SEQ ID NO: 78 - RNA, 40 nt, synthetic construct
  misc_feature 1..40: note = The sequence is synthetized.
  source 1..40: mol_type = other RNA
aatttctactattgtagattcacggattacgtggggtcta

SEQ ID NO: 79 - RNA, 40 nt, synthetic construct
  misc_feature 1..40: note = The sequence is synthetized.
  source 1..40: mol_type = other RNA
aatttctactattgtagatttggtattcctcccgtggctt

SEQ ID NO: 80 - RNA, 40 nt, synthetic construct
  misc_feature 1..40: note = The sequence is synthetized.
  source 1..40: mol_type = other RNA
aatttctactattgtagattgttacgtatccgatcacatt

SEQ ID NO: 81 - RNA, 40 nt, synthetic construct
  misc_feature 1..40: note = The sequence is synthetized.
  source 1..40: mol_type = other RNA
aatttctactattgtagatttcagccgtagtgatagaccc

SEQ ID NO: 82 - RNA, 40 nt, synthetic construct
  misc_feature 1..40: note = The sequence is synthetized.
  source 1..40: mol_type = other RNA
aatttctactattgtagattaaaggtaatcatcatcgcac

SEQ ID NO: 83 - RNA, 40 nt, synthetic construct
  misc_feature 1..40: note = The sequence is synthetized.
  source 1..40: mol_type = other RNA
aatttctactattgtagattgtatgtaagagctgcagaac

SEQ ID NO: 84 - RNA, 40 nt, synthetic construct
  misc_feature 1..40: note = The sequence is synthetized.
  source 1..40: mol_type = other RNA
aatttctactattgtagattaatataaaattctcttgctc

SEQ ID NO: 85 - RNA, 40 nt, synthetic construct
  misc_feature 1..40: note = The sequence is synthetized.
  source 1..40: mol_type = other RNA
aatttctactattgtagattcagtggtcatattaacgtat

SEQ ID NO: 86 - RNA, 40 nt, synthetic construct
  misc_feature 1..40: note = The sequence is synthetized.
  source 1..40: mol_type = other RNA
aatttctactattgtagattgggctaatatcagatataga

SEQ ID NO: 87 - RNA, 40 nt, synthetic construct
  misc_feature 1..40: note = The sequence is synthetized.
  source 1..40: mol_type = other RNA
aatttctactattgtagattctgtatctctatcctgaaag

SEQ ID NO: 88 - RNA, 40 nt, synthetic construct
  misc_feature 1..40: note = The sequence is synthetized.
  source 1..40: mol_type = other RNA
aatttctactattgtagattgttccaggtaggttttaatc

SEQ ID NO: 89 - RNA, 40 nt, synthetic construct
  misc_feature 1..40: note = The sequence is synthetized.
  source 1..40: mol_type = other RNA
aatttctactattgtagattctttgatggaaatttatcga

SEQ ID NO: 90 - RNA, 40 nt, synthetic construct
  misc_feature 1..40: note = The sequence is synthetized.
  source 1..40: mol_type = other RNA
aatttctactattgtagatttatgtaagagctgcagaact

SEQ ID NO: 91 - RNA, 40 nt, synthetic construct
  misc_feature 1..40: note = The sequence is synthetized.
  source 1..40: mol_type = other RNA
aatttctactattgtagattaatgtgatcggatacgtaac

SEQ ID NO: 92 - RNA, 40 nt, synthetic construct
  misc_feature 1..40: note = The sequence is synthetized.
  source 1..40: mol_type = other RNA
aatttctactattgtagatttagagatacagctcttccag

SEQ ID NO: 93 - RNA, 40 nt, synthetic construct
  misc_feature 1..40: note = The sequence is synthetized.
  source 1..40: mol_type = other RNA
aatttctactattgtagattgatgccgataccacaagatc

SEQ ID NO: 94 - RNA, 40 nt, synthetic construct
  misc_feature 1..40: note = The sequence is synthetized.
  source 1..40: mol_type = other RNA
aatttctactattgtagattacgggaggaataccaaccca

SEQ ID NO: 95 - RNA, 40 nt, synthetic construct
  misc_feature 1..40: note = The sequence is synthetized.
  source 1..40: mol_type = other RNA
aatttctactattgtagattgcagaactttgatggaaatt

SEQ ID NO: 96 - RNA, 40 nt, synthetic construct
  misc_feature 1..40: note = The sequence is synthetized.
  source 1..40: mol_type = other RNA
aatttctactattgtagattggattacgtggggtctatca

SEQ ID NO: 97 - RNA, 40 nt, synthetic construct
  misc_feature 1..40: note = The sequence is synthetized.
  source 1..40: mol_type = other RNA
aatttctactattgtagattacccttccactacggaggca

SEQ ID NO: 98 - RNA, 40 nt, synthetic construct
  misc_feature 1..40: note = The sequence is synthetized.
  source 1..40: mol_type = other RNA
aatttctactattgtagattctcttccagacgcatgttca

SEQ ID NO: 99 - RNA, 40 nt, synthetic construct
  misc_feature 1..40: note = The sequence is synthetized.
  source 1..40: mol_type = other RNA
aatttctactattgtagatttaatatgaccactgggttgg

SEQ ID NO: 100 - RNA, 40 nt, synthetic construct
  misc_feature 1..40: note = The sequence is synthetized.
  source 1..40: mol_type = other RNA
aatttctactattgtagattaggcaatgcgattaaaaccc

SEQ ID NO: 101 - RNA, 40 nt, synthetic construct
  misc_feature 1..40: note = The sequence is synthetized.
  source 1..40: mol_type = other RNA
aatttctactattgtagattatgttcatctatatctgata

SEQ ID NO: 102 - RNA, 40 nt, synthetic construct
  misc_feature 1..40: note = The sequence is synthetized.
  source 1..40: mol_type = other RNA
aatttctactattgtagattggatcatcgggggttttaat

SEQ ID NO: 103 - RNA, 40 nt, synthetic construct
  misc_feature 1..40: note = The sequence is synthetized.
  source 1..40: mol_type = other RNA
aatttctactattgtagatttgagcagttacggaaatgtt

SEQ ID NO: 104 - RNA, 40 nt, synthetic construct
  misc_feature 1..40: note = The sequence is synthetized.
  source 1..40: mol_type = other RNA
aatttctactattgtagattattattaaaaacatttccgt

SEQ ID NO: 105 - DNA, 141 bp, African swine fever virus
  source 1..141: mol_type = other DNA
ccatttaagagcagacattagtttttcatcgtggtggttattgttggtgtgggtcacctgcgttttatggacacgtatcagcgaaaagcgaacgcgttttacaaaaaggttgtgtatttcaggggttacaaacaggttatt

SEQ ID NO: 106 - DNA, 28 bp, synthetic construct
  misc_feature 1..28: note = The sequence is synthetized.
  source 1..28: mol_type = other DNA
ccatttaagagcagacattagtttttca

SEQ ID NO: 107 - DNA, 28 bp, synthetic construct
  misc_feature 1..28: note = The sequence is synthetized.
  source 1..28: mol_type = other DNA
ccatttatgagcagacattagtttttca

SEQ ID NO: 108 - DNA, 28 bp, synthetic construct
  misc_feature 1..28: note = The sequence is synthetized.
  source 1..28: mol_type = other DNA
ccatttaacagcagacattagtttttca

SEQ ID NO: 109 - DNA, 28 bp, synthetic construct
  misc_feature 1..28: note = The sequence is synthetized.
  source 1..28: mol_type = other DNA
ccatttaagtgcagacattagtttttca

SEQ ID NO: 110 - DNA, 28 bp, synthetic construct
  misc_feature 1..28: note = The sequence is synthetized.
  source 1..28: mol_type = other DNA
ccatttaagaccagacattagtttttca

SEQ ID NO: 111 - DNA, 28 bp, synthetic construct
  misc_feature 1..28: note = The sequence is synthetized.
  source 1..28: mol_type = other DNA
ccatttaagaggagacattagtttttca

SEQ ID NO: 112 - DNA, 28 bp, synthetic construct
  misc_feature 1..28: note = The sequence is synthetized.
  source 1..28: mol_type = other DNA
ccatttaagagctgacattagtttttca

SEQ ID NO: 113 - DNA, 28 bp, synthetic construct
  misc_feature 1..28: note = The sequence is synthetized.
  source 1..28: mol_type = other DNA
ccatttaagagcacacattagtttttca

SEQ ID NO: 114 - DNA, 28 bp, synthetic construct
  misc_feature 1..28: note = The sequence is synthetized.
  source 1..28: mol_type = other DNA
ccatttaagagcagtcattagtttttca

SEQ ID NO: 115 - DNA, 28 bp, synthetic construct
  misc_feature 1..28: note = The sequence is synthetized.
  source 1..28: mol_type = other DNA
ccatttaagagcagagattagtttttca

SEQ ID NO: 116 - DNA, 28 bp, synthetic construct
  misc_feature 1..28: note = The sequence is synthetized.
  source 1..28: mol_type = other DNA
ccatttaagagcagactttagtttttca

SEQ ID NO: 117 - DNA, 36 bp, synthetic construct
  misc_feature 1..36: note = The sequence is synthetized.
  source 1..36: mol_type = other DNA
ccatttaagagcagacaatagtttttcatcgtggtg

SEQ ID NO: 118 - DNA, 36 bp, synthetic construct
  misc_feature 1..36: note = The sequence is synthetized.
  source 1..36: mol_type = other DNA
ccatttaagagcagacataagtttttcatcgtggtg

SEQ ID NO: 119 - DNA, 36 bp, synthetic construct
  misc_feature 1..36: note = The sequence is synthetized.
  source 1..36: mol_type = other DNA
ccatttaagagcagacatttgtttttcatcgtggtg

SEQ ID NO: 120 - DNA, 36 bp, synthetic construct
  misc_feature 1..36: note = The sequence is synthetized.
  source 1..36: mol_type = other DNA
ccatttaagagcagacattactttttcatcgtggtg

SEQ ID NO: 121 - DNA, 36 bp, synthetic construct
  misc_feature 1..36: note = The sequence is synthetized.
  source 1..36: mol_type = other DNA
ccatttaagagcagacattagattttcatcgtggtg

SEQ ID NO: 122 - DNA, 36 bp, synthetic construct
  misc_feature 1..36: note = The sequence is synthetized.
  source 1..36: mol_type = other DNA
ccatttaagagcagacattagtatttcatcgtggtg

SEQ ID NO: 123 - DNA, 36 bp, synthetic construct
  misc_feature 1..36: note = The sequence is synthetized.
  source 1..36: mol_type = other DNA
ccatttaagagcagacattagttattcatcgtggtg

SEQ ID NO: 124 - DNA, 36 bp, synthetic construct
  misc_feature 1..36: note = The sequence is synthetized.
  source 1..36: mol_type = other DNA
ccatttaagagcagacattagtttatcatcgtggtg

SEQ ID NO: 125 - DNA, 36 bp, synthetic construct
  misc_feature 1..36: note = The sequence is synthetized.
  source 1..36: mol_type = other DNA
ccatttaagagcagacattagttttacatcgtggtg

SEQ ID NO: 126 - DNA, 36 bp, synthetic construct
  misc_feature 1..36: note = The sequence is synthetized.
  source 1..36: mol_type = other DNA
ccatttaagagcagacattagtttttgatcgtggtg

SEQ ID NO: 127 - DNA, 29 bp, synthetic construct
  misc_feature 1..29: note = The sequence is synthetized.
  source 1..29: mol_type = other DNA
caataacctgtttgtaacccctgaaatac

SEQ ID NO: 128 - DNA, 591 bp, Homo sapiens
  source 1..591: mol_type = other DNA
gccctacatctgctctccctccactaagaagaacctctttgtgtggcgaaagtaaaagtattagggcttttaagttgcccagagtcaaggaacacggataaagacgctgggagattgacatgcatttcgaccaatagcattgcagagaggcgtatcatttcgcggatgttccaatcagtacgcagagagtcgccgtctccaaggtgaaagcggaagtagggccttcgcgcacctcatggaatcccttctgcagcacctggatcgcttttccgagcttctggcggtctcaagcactacctacgtcagcacctgggaccccgccaccgtgcgccgggccttgcagtgggcgcgctacctgcgccacatccatcggcgctttggtcggcatggccccattcgcacggctctggagcggcggctgcacaaccagtggaggcaagagggcggctttgggcggggtccagttccgggattagcgaacttccaggccctcggtcactgtgacgtcctgctctctctgcgcctgctggagaaccgggccctcggggatgcagctcgttaccacctggtgcagcaactctttcccggccc

SEQ ID NO: 129 - RNA, 40 nt, synthetic construct
  misc_feature 1..40: note = The sequence is synthetized.
  source 1..40: mol_type = other RNA
aatttctactattgtagattgtcggcatggccccattcgc

SEQ ID NO: 130 - DNA, 601 bp, Homo sapiens
  source 1..601: mol_type = other DNA
catcaccaacccacagccaaggcggcgctggctttttttttttttttaatctttaacaatttgaatatttgtttttacaaaggtgcattttttaatagggcttggggagtcccagaggtatccagcagaggggagaagaaagagagatgtagggctagaggggtgaggctgaaacagtgacctgtcttggttttcgctccgaaggtaaaagaaatcattgagtcccccgccttcagaagagggtgcattttcaggaggaagcgatggcttcagacagcatatttgagtcatttccttcgtacccacagtgcttcatgagaggtgagtacatgctggtcttgtaatatctacttttgctcagctttgcctgtaatgaaatggcagcttgtttcacctcggtgcagagatgcctcggtgcctgccagttccctgtcttgtttgtgagaggaattcaaactgaggcatatgattacaagtctattggattacttactaatcagatggaagctcttcagaaatgttttaataaatacttagttatgctgttggagtgttcagtcggtgcgtgagaactttgtcaagtgcgagtaagttgtgctgg

SEQ ID NO: 131 - DNA, 694 bp, Homo sapiens
  source 1..694: mol_type = other DNA
ggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgg

SEQ ID NO: 132 - RNA, 40 nt, synthetic construct
  misc_feature 1..40: note = The sequence is synthetized.
  source 1..40: mol_type = other RNA
aatttctactattgtagatttggttgcccaccctagtcat

SEQ ID NO: 133 - RNA, 40 nt, synthetic construct
  misc_feature 1..40: note = The sequence is synthetized.
  source 1..40: mol_type = other RNA
aatttctactattgtagatttactttgtcctccggttctg

SEQ ID NO: 134 - DNA, 12 bp, synthetic construct
  misc_feature 1..12: note = The sequence is synthetized.
  source 1..12: mol_type = other DNA
gtatccagtgcg

SEQ ID NO: 135 - RNA, 19 nt, synthetic construct
  source 1..19: mol_type = other RNA
aatttctactattgtagat

SEQ ID NO: 136 - RNA, 19 nt, synthetic construct
  source 1..19: mol_type = other RNA
aatttctactgttgtagat

SEQ ID NO: 137 - RNA, 19 nt, synthetic construct
  source 1..19: mol_type = other RNA
aatttctactcttgtagat

SEQ ID NO: 138 - DNA, 41 bp, synthetic construct
  source 1..41: mol_type = other DNA
ccatttaagagcagacattagtttttcatcgtggtggttat

SEQ ID NO: 139 - DNA, 41 bp, synthetic construct
  source 1..41: mol_type = other DNA
ccatttatgagcagacattagtttttcatcgtggtggttat

SEQ ID NO: 140 - DNA, 41 bp, synthetic construct
  source 1..41: mol_type = other DNA
ccatttaacagcagacattagtttttcatcgtggtggttat

SEQ ID NO: 141 - DNA, 41 bp, synthetic construct
  source 1..41: mol_type = other DNA
ccatttaagtgcagacattagtttttcatcgtggtggttat

SEQ ID NO: 142 - DNA, 41 bp, synthetic construct
  source 1..41: mol_type = other DNA
ccatttaagaccagacattagtttttcatcgtggtggttat

SEQ ID NO: 143 - DNA, 41 bp, synthetic construct
  source 1..41: mol_type = other DNA
ccatttaagaggagacattagtttttcatcgtggtggttat

SEQ ID NO: 144 - DNA, 41 bp, synthetic construct
  source 1..41: mol_type = other DNA
ccatttaagagctgacattagtttttcatcgtggtggttat

SEQ ID NO: 145 - DNA, 41 bp, synthetic construct
  source 1..41: mol_type = other DNA
ccatttaagagcacacattagtttttcatcgtggtggttat

SEQ ID NO: 146 - DNA, 41 bp, synthetic construct
  source 1..41: mol_type = other DNA
ccatttaagagcagtcattagtttttcatcgtggtggttat

SEQ ID NO: 147 - DNA, 41 bp, synthetic construct
  source 1..41: mol_type = other DNA
ccatttaagaccagagattagtttttcatcgtggtggttat

SEQ ID NO: 148 - DNA, 41 bp, synthetic construct
  source 1..41: mol_type = other DNA
ccatttaagaggagactttagtttttcatcgtggtggttat

SEQ ID NO: 149 - DNA, 41 bp, synthetic construct
  source 1..41: mol_type = other DNA
ccatttaagagcagacaatagtttttcatcgtggtggttat

SEQ ID NO: 150 - DNA, 41 bp, synthetic construct
  source 1..41: mol_type = other DNA
ccatttaagagcagacataagtttttcatcgtggtggttat

SEQ ID NO: 151 - DNA, 41 bp, synthetic construct
  source 1..41: mol_type = other DNA
ccatttaagagcagacatttgtttttcatcgtggtggttat

SEQ ID NO: 152 - DNA, 41 bp, synthetic construct
  source 1..41: mol_type = other DNA
ccatttaagagcagacattactttttcatcgtggtggttat

SEQ ID NO: 153 - DNA, 41 bp, synthetic construct
  source 1..41: mol_type = other DNA
ccatttaagagcagacattagattttcatcgtggtggttat

SEQ ID NO: 154 - DNA, 41 bp, synthetic construct
  source 1..41: mol_type = other DNA
ccatttaagagcagacattagtatttcatcgtggtggttat

SEQ ID NO: 155 - DNA, 41 bp, synthetic construct
  source 1..41: mol_type = other DNA
ccatttaagagcagacattagttattcatcgtggtggttat

SEQ ID NO: 156 - DNA, 41 bp, synthetic construct
  source 1..41: mol_type = other DNA
ccatttaagagcagacattagtttatcatcgtggtggttat

SEQ ID NO: 157 - DNA, 41 bp, synthetic construct
  source 1..41: mol_type = other DNA
ccatttaagagcagacattagttttacatcgtggtggttat

SEQ ID NO: 158 - DNA, 41 bp, synthetic construct
  source 1..41: mol_type = other DNA
ccatttaagagcagacattagtttttgatcgtggtggttat

(19) United States
(12) Patent Application Publication
(10) Pub. No.: US 2024/0368675 A1
(43) Pub. Date: Nov. 7, 2024

(54) PAM-LESS ENDONUCLEASE AND GENE EDITING SYSTEM MEDIATED BY PAM-LESS ENDONUCLEASE

(71) Applicant: HUAZHONG AGRICULTURAL UNIVERSITY, Wuhan (CN)

(72) Inventors: Shuhong ZHAO, Wuhan (CN); Shengsong XIE, Wuhan (CN); Xinyun LI, Wuhan (CN); Bingrong XU, Wuhan (CN); Sheng LI, Wuhan (CN); Dagang TAO, Wuhan (CN); Jinxue RUAN, Wuhan (CN); Yuan WANG, Wuhan (CN); Hailong LIU, Wuhan (CN)

(73) Assignee: HUAZHONG AGRICULTURAL UNIVERSITY, Wuhan (CN)

(21) Appl. No.: 18/764,397

(22) Filed: Jul. 5, 2024

Related U.S. Application Data
(63) Continuation of application No. PCT/CN2023/137462, filed on Dec. 8, 2023.

(30) Foreign Application Priority Data
Jan. 2, 2023 (CN) 202310006257.7

(51) Int. Cl. C12Q 1/6818; C12N 9/22; C12N 15/11; C12N 15/90

(52) U.S. Cl. CPC C12Q 1/6818; C12N 9/22; C12N 15/11; C12N 15/907; C12N 2310/20

(57) ABSTRACT

A PAM-less CRISPR/Cas gene editing system is provided. Specifically, the PAM-less CRISPR/Cas gene editing system offers Gs12-10, a PAM-less endonuclease identified through functional metagenomic mining approach, which can cleave target DNA at nearly any position in the genome. This technology enables a nucleic acid visualization detection and genome editing platform mediated by the CRISPR/Gs12-10 system, holding broad application potential in genome-modification and nucleic acid detection fields.

### CROSS REFERENCE TO THE RELATED APPLICATIONS

This application is a continuation application of International Application No. PCT/CN2023/137462, filed on Dec. 8, 2023, which is based upon and claims priority to Chinese Patent Application No. 202310006257.7, filed on Jan. 2, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBWHYC007_Sequence_Listing(20240719113418).xml, created on Jul. 19, 2024, and is 216,247 bytes in size.

### TECHNICAL FIELD

The present invention relates to the field of genome editing technology, specifically involving the development and application of newly identified PAM-less endonuclease Gs12-10 and nucleic acid detection, as well as genome editing technology mediated by the newly identified PAM-less endonuclease Gs12-10.

### BACKGROUND

Clustered regularly interspaced short palindromic repeats (CRISPR)/CRISPR-associated protein 9 (Cas9) is the third-generation gene editing technology, following the introduction of gene editing technologies such as ZFN and TALENs. The gene editing technology has become popular worldwide after nearly 10 years of development, becoming one of the most efficient, simple, cost-effective, and easy-to-operate technologies in existing gene editing and genome modification. This technology has shown infinite potential in basic research, clinical transformation, and agricultural production. The CRISPR/Cas system is a natural immune system in prokaryotes, which comprises two parts: CRISPR locus and Cas gene (CRISPR-associated gene). At present, CRISPR/Cas systems can be divided into two categories. The first category is that their effector factors for cleaving exogenous nucleic acids are complexes formed by multiple Cas proteins, including type I, type III, and type IV Cas proteins; the second category is that their action factors are relatively single Cas proteins, such as type II Cas9 protein and type V Cas12a protein.

The CRISPR/Cas9 or Cas12a system is mainly composed of Cas9 protein or Cas12a protein and guide RNA (sgRNA or crRNA). Among them, sgRNA provides sequence specificity, targeting the paired DNA sequence, thereby providing precise localization for Cas9 nuclease or Cas12a nuclease and ultimately cleaving DNA, thereby achieving gene editing. In addition to sgRNA, CRISPR/Cas9 or Cas12a also relies on recognizing the sequences of protospacer adjacent motifs (PAM) on the target DNA when performing editing functions. At present, the most widely used CRISPR system is type II CRISPR/Cas system. In addition to CRISPR/Cas9, there are also CRISPR/Cas12, CRISPR/Cas13, and CRISPR/Cas14. Among them, the PAM sequence recognized by SpCas9 nuclease is “NGG”, while the PAM sequence recognized by Cas12a nuclease is “TTTV or TTV”. The complexity of the PAM sequence determines the upper limit of editable sites. In practical applications, the lack of PAM sequence at the target site often leads to the inability of Cas9 or Cas12a to target, thereby hindering the effectiveness of gene editing. Therefore, exploring loose PAM (PAM less) nucleases has become a research hotspot.

For a long time, researchers have been committed to optimizing and upgrading Cas9 or Cas12 proteins to expand their compatibility to different PAM sequences, especially allowing Cas proteins to have genome-wide editing capabilities. Taking SpCas9 as an example: in 2015, SpCas9-VRQR mutants that can recognize NGA and SpCas9-VRER mutants that can recognize NGCG were obtained through an error-prone PCR strategy. In 2018, xCas9 3.7 variants were constructed using directed evolution technology PACE to recognize NGG, NG, GAA, and GAT; In the same year, a more active SpCas9-NG variant was developed, and its recognized PAM sequence was extended to NG. In 2020, using PACE technology, a series of PAM sequences recognized by SpCas9 mutants were further expanded to NRNH (R is A/G, H is A/C/T). This series of work has almost freed SpCas9 and its mutants from PAM troubles. In 2020, the SpCas9 protein was modified to develop SpRY whose recognized PAM sequences covering NRN and NYN (Y is C/T) (NRN>NYN). However, there is currently no PAM less Cas12a nuclease.

Compared with Cas9, Cas12a has many advantages, such as shorter sgRNA or crRNA, making it easier to be delivered to cells; After cleaving, cohesive ends are generated, which is more conducive to precise genome recognition and editing; The distance between the cleaving site and its recognition site is relatively far, which can achieve the purpose of continuous multiple edits. In addition, the greatest feature of the Cas12a protein lies in that it is not only used for gene editing at the cellular or individual level, but also widely used for highly sensitive and specific detection of small molecules such as nucleic acids or proteins (reference patent ZL202010060317.X). Research has found that once Cas12a forms a ternary complex with crRNA and target DNA, the complex exhibits strong “random cleavage” activity and cleaves any single-stranded DNA in the system into fragments (known as trans-cleavage or cis-cleavage). At present, there are relatively few known Cas12a proteins, such as natural AsCas12a, LbCas12a, and FnCas12a, as well as artificially modified enhanced enAsCas12a, whose recognized PAM sequences are all “TTTV or TTV”, resulting in a small target recognition range.

For a long time, researchers have been committed to optimizing and upgrading Cas9 or Cas12a proteins to expand their compatibility with different PAM sequences, and hope to develop new Cas proteins without being restricted by PAM sequences, allowing the CRISPR/Cas system to have genome-wide editing capabilities. Although studies have shown that there are differences in the PAM sequences of Cas9 or Cas12a proteins from different bacterial sources, the existence of natural and active PAM-less Cas12a proteins has not been reported yet.

Therefore, there is still an urgent need in this field to search for CRISPR/Cas12a gene editing systems without being restricted by PAM sequences.

### SUMMARY

The present invention has developed for the first time a CRISPR/Gs12-10 gene editing system without PAM sequence requirements, which can cleave target DNA at almost any position in the genome. The present invention also establishes a nucleic acid visualization detection and genome editing technology mediated by Gs12-10 protein.

To achieve the above objectives, the present invention comprises the following technical solutions:

The endonucleases in the CRISPR/Cas system comprise the following:
I. The Gs12-10 protein of the amino acid sequence shown in SEQ ID NO: 1;
II. A protein with more than 80% sequence similarity compared with the amino acid sequence shown in SEQ ID NO: 1, and basically retaining the biological function of the sequence from which it derives;
III. A protein with one or more amino acid substitutions, deletions, or additions compared with the amino acid sequence shown in SEQ ID NO: 1, and basically retaining the biological function of the sequence from which it derives.

Fusion proteins, comprising the endonucleases as above and peptides connected to the N-terminus or C-terminus of the proteins.

Polynucleotides, encoding the endonucleases or the above fusion proteins.

Vectors or host cells comprising the polynucleotides.

The application of the endonucleases as above in gene editing includes modifying genes, knocking out genes, altering the expression of gene products, repairing mutations, or inserting polynucleotides in prokaryotic genomes, eukaryotic genomes, or in vitro genes.

A CRISPR/Cas gene editing system comprising the endonucleases, fusion proteins, polynucleotides, vectors, or host cells as above. Furthermore, it also comprises direct repeat sequences that can bind to the endonucleases as above and guiding sequences that can target the target sequence.

A visual nucleic acid detection kit comprising the endonucleases as above, single-stranded DNA fluorescence quenching reporters, and guide RNA paired with target nucleic acids.

The technical solution of the present invention has the following main beneficial effects:
1. The present invention provides for the first time a novel member of the CRISPR/Cas12a system family, Gs12-10, discovered by combining metagenomics mining approach and experimental methods.
2. The present invention discovers a PAM-less endonuclease Gs12-10, which has the advantage of being able to cleave target DNA at almost any position in the genome, greatly expanding the coverage of gene editing targets.
3. The present invention provides for the first time a nucleic acid visualization detection and genome editing technology mediated by the CRISPR/Gs12-10 system.

### BRIEF DESCRIPTION OF THE DRAWINGS

FIGS. 1A-1B. Prediction of guide RNA-dependent endonuclease Gs12-10 using metagenomic mining approach (FIG. 1A) and phylogenetic tree analysis (FIG. 1B).

FIGS. 2A-2B. DR sequence pattern diagram of endonuclease Gs12-10 locus, domain, and guide RNA. FIG. 2A. Schematic diagram of the Gs12-10 locus; FIG. 2B. The secondary structure folding and multiple sequence alignments of the DR sequence of the guide RNA, Gs12-10: SEQ ID NO: 135, LbCas12a and FnCas12a: SEQ ID NO: 136, AsCas12a: SEQ ID NO: 137.

FIG. 3. Conservative analysis of predicted amino acid sequences of Gs12-10 protein and known amino acid sequences of Cas12a proteins (AsCas12a, LbCas12a, and FnCas12a).

FIG. 4. Detection of the activity of Gs12-10 cleaving double-stranded DNA target by gel electrophoresis. The target is the amplified fragment of ASFV p72 gene of African swine fever virus, and the recognized target site PAM is “TTTV”.

FIGS. 5A-5B. Identification of the characteristics of recognition of PAM by Gs12-10 using PAM library subtraction experiment in bacteria.

FIGS. 6A-6B. Validation of in vitro cleavage ability of Gs12-10 towards the same target site containing different PAMs in linear double-stranded DNA. The target is an amplified fragment of the ASFV p72 gene of African swine fever virus, with the same spacer sequence but different PAM sequences.

FIGS. 7A-7B. Validation of in vitro cleavage ability of Gs12-10 towards different target sites containing different PAMs in circular plasmid DNA. The target plasmid is puc19, and there are three types of bands: nicked, linear, and supercoiled, with different electrophoretic migration rates. If the plasmid is cut, it will transition from a supercoil to a linear shape, causing a change in the size of the migration band.

FIGS. 8A-8C. Comparison of the cis-cleavage activity of Gs12-10 and enhanced enAsCas12a towards ssDNA-FQ reporting system base preference. The target is the amplified fragment of the ASFV p72 gene of African swine fever virus, and the recognized target site PAM is “TTTV”. FIG. 8A. Blue light instrument detection results; FIGS. 8B-8C. Multimode reader detection results.

FIG. 9. The optimal enzyme digestion temperature for evaluating the cis-cleavage activity of Gs12-10. The target is the ASFV p72 gene.

FIGS. 10A-10C. Validation of the cis-cleavage activity of Gs12-10 towards different target sites containing different PAMs in circular plasmid DNA. FIG. 10A. Experimental flow diagram, FIG. 10B. Blue light instrument detection results, FIG. 10C. Multimode reader detection results.

FIGS. 11A-11B. Validation of the cis-cleavage activity of Gs12-10 towards the same target site containing different PAMs in linear double-stranded DNA. The target is an amplified fragment of the ASFV p72 gene of African swine fever virus, with the same spacer sequence but different PAM sequences. FIG. 11A. Experimental procedure diagram, FIG. 11B. Blue light instrument detection results.

FIGS. 12A-12C. Validation of the cis-cleavage activity of Gs12-10 towards different target sites containing different PAMs in linear double-stranded DNA. The target is the amplified fragment of the ASFV p72 gene of African swine fever virus. FIG. 12A. Experimental flow diagram, FIG. 12B. Blue light instrument detection results, FIG. 12C. Multimode reader detection results.

FIGS. 13A-13B. Evaluation of the positional effect of single base mismatch on the Gs12-10 cis-cleavage activity in the target (“PC” and “Target Sequences 1-20” are shown in SEQ ID NO: 138-SEQ ID NO: 158). The target is the amplified fragment of the ASFV p72 gene of African swine fever virus, with PC as the positive control and NC as the negative control.

FIGS. 14A-14B. Detection of genome editing activity of RNP delivered Gs12-10 protein and in vitro transcribed crRNA complex in cells through T7EN1 enzyme digestion assay. The target is the human FANCF gene, and Control is a negative control.

FIGS. 15A-15B. Detection of genome editing activity of single or tandem crRNA expression vectors co-transfected with liposomes into Gs12-10 eukaryotic expression vector in cells through T7EN1 enzyme digestion assay. FIG. 15A. Schematic diagram of a single or tandem crRNA expression vector. FIG. 15B. T7EN1 enzyme digestion experiment. The cell is human HEK293T.

FIGS. 16A-16B. Evaluation of CRISPR/Gs12-10 system-mediated multiple gene editing activity in eukaryotic cells. FIG. 16A. Pattern diagram of tandem crRNA expression vector; FIG. 16B. T7EN1 enzyme digestion experiment. The cell is human HEK293T.

### DETAILED DESCRIPTION OF THE EMBODIMENTS

### Terminology

Unless defined otherwise, all technical and scientific terms used herein have the same meaning as commonly understood by one of ordinary skill in the art to which this invention belongs.

Genie scissor (Lingjian) is a family of endonucleases, where Genie means elf, representing bacterial origin, and scissor represents gene scissors, indicating their potential gene editing functions. The Chinese name corresponding to Genie scissor endonuclease is “Lingjian” endonuclease, and the Genie scissor gene editing system represents the gene editing system mediated by “Lingjian” endonuclease, abbreviated as “Lingjian gene editing”.

The protospacer adjacent motif (PAM) is a short DNA sequence (usually the length of 2-6 base pairs). The traditional view is that PAM is necessary for Cas nuclease cleavage, typically 3-4 nucleotides downstream of the cleavage site. Many different Cas endonucleases can be purified from different bacteria, and each enzyme may recognize different PAM sequences.

The present invention will be further described in combination with specific embodiments. It should be understood that these embodiments are only used to illustrate the invention and not to limit the scope of the invention.

### Example 1. Discovery of Novel Guide RNA-Dependent Endonucleases Through a Metagenomics-Based Mining Approach

Based on the bioinformatics identification process of a novel guide RNA-dependent endonuclease constructed by the inventor, deep mining of bacterial encoded proteins was carried out on the massive metagenomic sequencing data from public databases such as the Non-Redundant Protein Sequence Database (NCBI nr) and the Global Microbial Gene Catalog Database (GMGC). The general analysis process was as follows: for all contig sequences in the target database, use minced software to search and locate the CRISPR array, then use prodigal software to predict CRISPR array adjacent expressed proteins, use CD-hit software to remove redundancy from all predicted proteins, use mega software for protein clustering analysis, and use hmmer software to identify and classify CRISPR Cas similarity proteins. Finally, a new unknown bacterial protein was obtained, with an amino acid sequence as shown in SEQ ID NO: 1 and a nucleic acid sequence as shown in SEQ ID NO: 2.

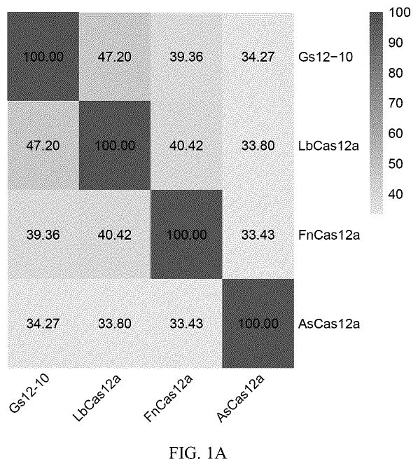

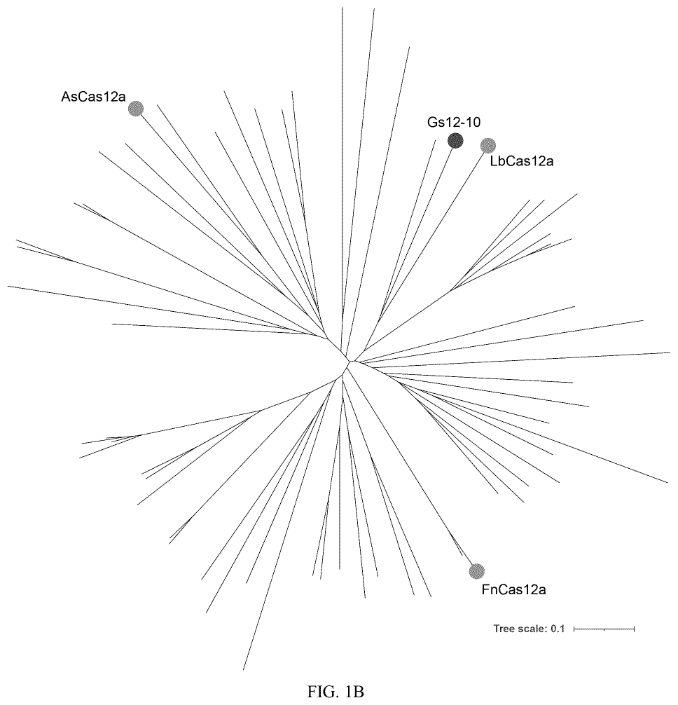

Through phylogenetic tree analysis, it is found that this new bacterial protein is located on different CRISPR-Cas12a phylogenetic branches (FIG. 1B), suggesting that it may be a new RNA-guided endonuclease. The inventor named the newly discovered proteins from different bacteria as Genie Scissor (GS) endonucleases. For the convenience of subsequent research, based on the origin of bacterial species, the inventor named this new unknown bacterial protein Gs12-10, following the naming convention of “endonuclease+numerical number”.

Next, the inventor utilized a localized blast program to perform sequence similarity alignment between this newly discovered bacterial protein and the NCBI nr database. The results show that the amino acid sequence conservation of the new Gs12-10 protein with known endonucleases LbCas12a, FnCas12a, and AsCas12a are 47.2%, 39.36%, and 34.27%, respectively (FIG. 1A).

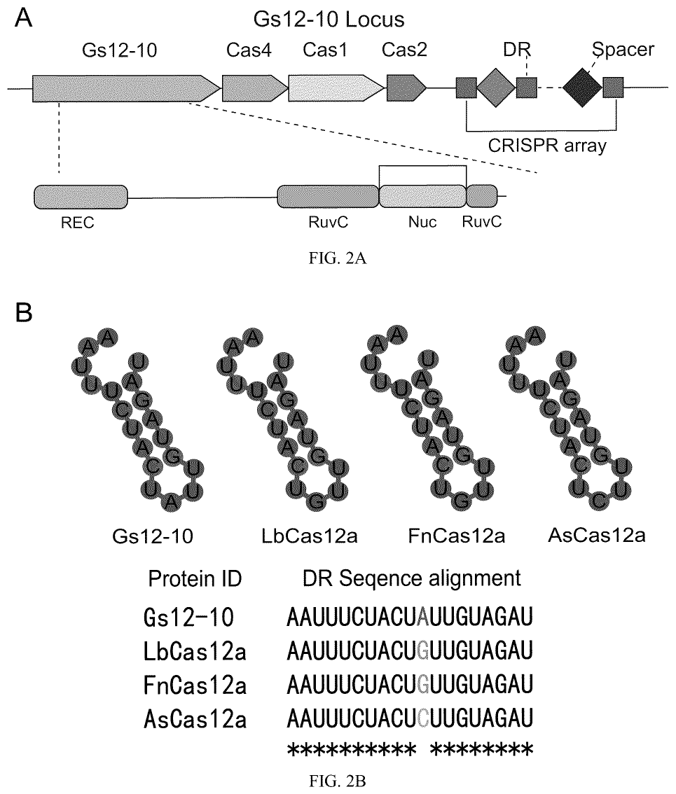

Furthermore, the inventor analyzed the loci of this protein using CRISPRCasFinder software. It is found that Gs12-10 has a CRISPR array sequence, which comprises multiple repeat and interval sequences, as well as Cas4, Cas1, and Cas2 proteins. By using hmmer software to perform hidden Markov model alignment analysis on domain sequences in Pfam database, REC1 domain (Alpha helical recognition lobe domain), RuvC nuclease domain, and NUC domain (Nuclease domain) were obtained, and it is speculated that this new bacterial protein may have nucleic acid cleavage activity; next, the inventor predicted and aligned multiple sequences of the DR sequence secondary structure of Gs12-10 through the RNAfold web server (http://rna.tbi.univie.ac.at/cgi-bin/RNAWebSuite/RNAfold.cgi) online website, and it is found that the newly predicted bacterial protein has a DR secondary structure similar to the known Cas12a protein, but with one base difference (FIGS. 2A-2B).

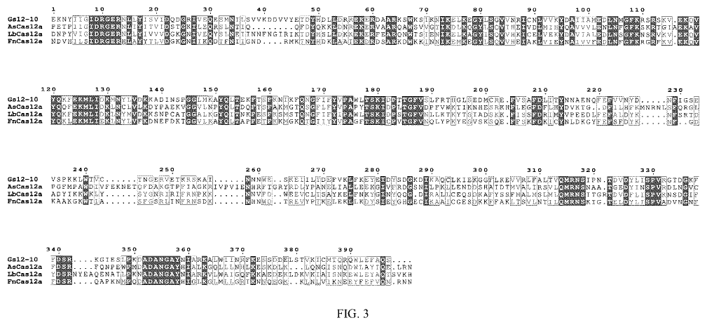

Finally, the inventor performed amino acid multiple sequence alignments of the RuvC and Nuc domains of Gs12-10 with known LbCas12a, FnCas12a, and AsCas12a proteins, respectively. As shown in FIG. 3, it is found that there is a significant difference in the amino acid sequence similarity between the structure of the Gs12-10 protein and the known Cas12a protein. Therefore, further experiments are urgently needed to determine whether it has nucleic acid-directed cleavage activity.

### Example 2. In Vitro Analysis of Guide RNA-Dependent Endonuclease Gs12-10's Nucleic Acid Cleavage Activity

This embodiment tested the cleavage activity of the Gs12-10 protein on double-stranded DNA through in vitro experiments. The guide RNA paired with the target nucleic acid was used to guide the recognition and binding of the Gs12-10 protein to the target nucleic acid, thereby stimulating the cleavage activity of the Gs12-10 protein towards the target nucleic acid and cleaving the double-stranded target nucleic acid in the system. Then, agarose gel electrophoresis was used to observe the size change of the target band to identify its enzyme digestion activity.

In this embodiment, the selected target double-stranded DNA (dsDNA) is the African swine fever P72 gene, PAM is TTTA, its sequence: CTGTAACGCAGCACAGCTGAACCGTTCTGAAGAAGAAGAAAGTTAATAGCAGATGCC GATACCACAAGATCAGCCGTAGTGATAGACCCCACGTAATCCGTGTCCCAACTAATAT AAAATTCTCTTGCTCTGGATACGTTAATATGACCACTGGGTTGGTATTCCTCCCGTGGC TTCAAAGCAAAGGTAATCATCATCGCACCCGGATCATCGGGGGTTTTAATCGCATTGC CTCCGTAGTGGAAGGGTATGTAAGAGCTGCAGAACTTTGATGGAAATTTATCGATAAG ATTGATACCATGAGCAGTTACGGAAATGTTTTTAATAATAGGTAATGTGATCGGATACG TAACGGGGCTAATATCAGATATAGATGAACATGCGTCTGGAAGAGCTGTATCTCTATCC TGAAAGCTTATCTCTGCGTGGTGAGTGGGCTGCATAATGGCGTTAACAACATGTCCGA ACTTGTGCCAATCTCGGTGTTGATGAGGATTTTGATCGGAGATGTTCCAGGTAGGTTT TAATCCTATAAACATATATTCAATGGGCCATTTAAGAGCAGACATTAGTTTTTCATCGT GGTGGTTATTGTTGGTGTGGGTCACCTGCGTTTTATGGACACGTATCAGCGAAAAGCG AACGCGTTTTACAAAAAGGTTGTGTATTTCAGGGGTTACAAACAGGTTATTGATGTAA AGTTCATTATTCGTGAGCGAGATTTCATTAATGACTCCTGGGATAAACCATGG (SEQ ID NO: 5); the bold marking is PAM, and the underline represents the target sequence. The guide RNA sequence is: AAUUUCUACUAUUGUAGAUUAGAGCAGACAUUAGUUUUUC (SEQ ID NO: 42) (the underlined area is the target area). Using the pmd-18t-p72 plasmid as a template, p72-F: CTGTAACGCAGCACAGCTGA (SEQ ID NO: 11), and p72-R: CCATGGTTTATCCCAGGAGT (SEQ ID NO: 14) as primers, PCR amplification was performed to obtain P72 double-stranded DNA. Secondly, the DNA sequence encoding Gs12-10 was synthesized by optimizing the Escherichia coli codon, and NLS nuclear localization signals were added to its C-terminus. The DNA sequence is shown in SEQ ID NO: 3. Subsequently, it was connected to the prokaryotic expression vector pET-28a and transformed into the Escherichia coli BL21 strain. After identifying the positive clone, IPTG-induced expression was performed, and the target protein was purified through affinity chromatography. The following system was adopted in the in vitro cleaving reaction: 10×CutSmart Buffer 2 μL. The predicted Gs12-10-NLS-tagged protein was 500 ng, the guide RNA was 500 ng, and the P72 target amplification product was 2 μL. The system was incubated at 37° C. for 0.5 min, 2 min, 10 min, and 20 min, respectively. After the reaction was completed, 1 μL of protease K was added separately and incubated at 55° C. for 10 min to terminate the reaction. Guide RNA and target nucleic acid were added in the experimental group, while guide RNA was not added in the control group. After the reaction, 1% agarose gel electrophoresis was carried out, the difference between the target bands of the new endonuclease Gs12-10 predicted under different reaction times in the experimental group and the control group was detected by imaging observation under UV transilluminator, and the cleaving efficiency was analyzed through Image J software.

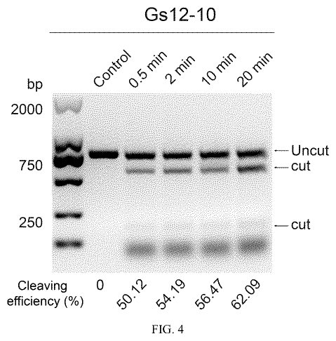

As shown in FIG. 4, compared with the control group without guide RNA, the Gs12-10 protein in the experimental group can cleave the target double-stranded DNA in the reaction solution in just 0.5 min, with two distinct cleavage bands. The cleaving efficiency is 50.12%. It is found that as the reaction time increases, the cleaving efficiency also significantly improves, reaching 54.19%, 56.47%, and 62.09%, respectively. From this, it can be seen that bacterial proteins predicted through metagenomic strategies have high nucleic acid-targeted cleavage activity as expected.

### Example 3. Identification of the Gs12-10 Protein, which Possesses Targeted Cleavage Ability without PAM Restrictions

The PAM sequence recognized by the Gs12-10 protein with low homology and in vitro target nucleic acid cleavage activity was identified through the bacterial PAM library subtraction experiment. Among them, the construction process of the randomly mixed PAM vector library is as follows: the DNA oligo sequence GGCCAGTGAATTCGAGCTCGGTACCCGGGNNNNNNNGAGAAGTCATTAATAAGGCC ACTGTTAAAAAGCTTGGCGTAATCATGGTCATAGCTGTTT (SEQ ID NO: 6) was synthesized, where N is a random deoxyribonucleotide. Oligo F: GGCCAGTGAATTCGAGCTCGG (SEQ ID NO: 7) and Oligo R: AAACAGCTATGACCATGATTACGCCAA (SEQ ID NO: 8) were used as upstream and downstream primers for PCR amplification; they were then connected to the pUC19 vector through homologous recombination. After transformation into Escherichia coli, the plasmid was extracted to form a random mixed PAM vector library. The guide RNA sequence used is: AAUUUCUACUAUUGUAGAUUGAGAAGUCAUUUAAUAAGGCCACU (SEQ ID NO: 9) (the underlined area is the target recognition sequence).

The bacterial PAM library subtraction experiment: the constructed vector pACYC-Duet-1-Gs12-10-crRNA co-expressing the predicted Gs12-10 protein and crRNA was transformed into the DE3 (BL21) competent cells to prepare a stable expression bacterial strain. The stable transgenic bacterial strain constructed from the expression vector pACYC-Duet-1-Gs12-10 without crRNA was used as a negative control. 100 ng of PAM library plasmids were transferred into stable expression bacterial strains, and screened using ampicillin and chloramphenicol-resistant plates. After 16 hours, the colonies on the plates were scraped off for plasmid extraction. Using 100 ng of extracted plasmids as templates, PCR amplification was performed using library sequencing primers Seq-F: GGCCAGTGAATTCGAGCTCGG (SEQ ID NO: 7) and PAM Seq-R: CAATTTCACACAGGAAACAGCTATGACC (SEQ ID NO: 10). After product recovery, the experimental and control groups were subjected to second-generation high-throughput sequencing, and the sequencing results were analyzed and displayed using Weblogo 3.0.

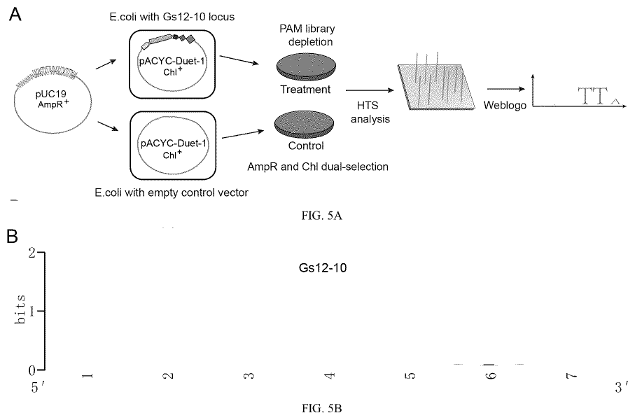

Identification of PAM sequence characteristics recognized by the Gs12-10: 16384 different types of PAM sequences contained in the starting vector library were counted for their frequency of occurrence in high-throughput sequencing in the experimental group and control group, and standardized using the total number of PAM sequences in each group. The calculation method for each PAM consumption change is log 2 (control group standardized value/experimental group standardized value). When this value is greater than 3.5, it is considered that this PAM has been significantly consumed. Then, Weblogo 3.0 was used to visually display the frequency of occurrence of significantly consumed PAM sequences at various positions. As shown in FIGS. 5A-5B, it is found that the Gs12-10 protein has an in vitro target cleavage ability without being restricted by PAM, indicating that its target recognition range greatly exceeds the known Cas12a protein with “TTTV” as PAM.

To demonstrate the reliability of the bacterial PAM library subtraction experiment, it was validated through in vitro enzyme digestion of double-stranded DNA. Using the pmd-18t-p72 plasmid as a template, P72 fragment 1 was amplified using P72-F1 and P72-R1 as primers. P72 fragment 2 was amplified using different P72-F2 and P72-R2 primers, and P72 fragment 3 was amplified using P72-F3 and P72-R3 primers. Finally, using P72-F1 and P72-R3 primers, fragments 1, 3, and different fragments 2 as templates, Overlap PCR was performed to obtain different PAM target double-stranded DNA (dsDNA) African swine fever P72 gene. The primer sequence is shown in the table below:

| | | SEQ |
|---|---|---|
| | | ID |
| Primer name | Sequence (5′-3′) | NO: |
| P72-F1 | CTGTAACGCAGCACAGCTGA | 11 |
| P72-R1 | CATATATTCAATGGGCCA | 12 |
| P72-F3 | ATCGTGGTGGTTATTGT | 13 |
| P72-R3 | CCATGGTTTATCCCAGGAGT | 14 |
| P72-R2 | TTTCGCTGATACGTGTCC | 15 |
| P72-F2-AATA | CATATATTCAATGGGCCAAATAAGAGCAGACATTAGT | 16 |
| | TTTTCATCGTGGTGGTTATTGT | |
| P72-F2-AATT | CATATATTCAATGGGCCAAATTAGAGCAGACATTAGT | 17 |
| | TTTTCATCGTGGTGGTTATTGT | |
| P72-F2-AATG | CATATATTCAATGGGCCAAATGAGAGCAGACATTAGT | 18 |
| | TTTTCATCGTGGTGGTTATTGT | |
| P72-F2-AATC | CATATATTCAATGGGCCAAATCAGAGCAGACATTAGT | 19 |
| | TTTTCATCGTGGTGGTTATTGT | |
| P72-F2-AGTA | CATATATTCAATGGGCCAAGTAAGAGCAGACATTAGT | 20 |
| | TTTTCATCGTGGTGGTTATTGT | |
| P72-F2-AGTT | CATATATTCAATGGGCCAAGTTAGAGCAGACATTAGT | 21 |
| | TTTTCATCGTGGTGGTTATTGT | |
| P72-F2-AGTG | CATATATTCAATGGGCCAAGTGAGAGCAGACATTAGT | 22 |
| | TTTTCATCGTGGTGGTTATTGT | |
| P72-F2-AGTC | CATATATTCAATGGGCCAAGTCAGAGCAGACATTAGT | 23 |
| | TTTTCATCGTGGTGGTTATTGT | |
| P72-F2-ACTA | CATATATTCAATGGGCCAACTAAGAGCAGACATTAGT | 24 |
| | TTTTCATCGTGGTGGTTATTGT | |
| P72-F2-ACTT | CATATATTCAATGGGCCAACTTAGAGCAGACATTAGT | 25 |
| | TTTTCATCGTGGTGGTTATTGT | |
| P72-F2-ACTG | CATATATTCAATGGGCCAACTGAGAGCAGACATTAGT | 26 |
| | TTTTCATCGTGGTGGTTATTGT | |
| P72-F2-ACTC | CATATATTCAATGGGCCAACTCAGAGCAGACATTAGT | 27 |
| | TTTTCATCGTGGTGGTTATTGT | |
| P72-F2-ATTA | CATATATTCAATGGGCCAATTAAGAGCAGACATTAGT | 28 |
| | TTTTCATCGTGGTGGTTATTGT | |
| P72-F2-ATTT | CATATATTCAATGGGCCAATTTAGAGCAGACATTAGT | 29 |
| | TTTTCATCGTGGTGGTTATTGT | |
| P72-F2-ATTG | CATATATTCAATGGGCCAATTGAGAGCAGACATTAGT | 30 |
| | TTTTCATCGTGGTGGTTATTGT | |
| P72-F2-ATTC | CATATATTCAATGGGCCAATTCAGAGCAGACATTAGT | 31 |
| | TTTTCATCGTGGTGGTTATTGT | |
| P72-F2-CCCC | CATATATTCAATGGGCCACCCAAGAGCAGACATTAGT | 32 |
| | TTTTCATCGTGGTGGTTATTGT | |
| P72-F2-TTTA | CATATATTCAATGGGCCATTTAAGAGCAGACATTAGT | 33 |
| | TTTTCATCGTGGTGGTTATTGT | |
| P72-F2-CGCC | CATATATTCAATGGGCCACGCCAGAGCAGACATTAGT | 34 |
| | TTTTCATCGTGGTGGTTATTGT | |
| P72-F2-CGGC | CATATATTCAATGGGCCACGGCAGAGCAGACATTAGT | 35 |
| | TTTTCATCGTGGTGGTTATTGT | |
| P72-F2-CCGC | CATATATTCAATGGGCCACCGCAGAGCAGACATTAGT | 36 |
| | TTTTCATCGTGGTGGTTATTGT | |
| P72-F2-GCCC | CATATATTCAATGGGCCAGCCCAGAGCAGACATTAGT | 37 |
| | TTTTCATCGTGGTGGTTATTGT | |
| P72-F2-GCGC | CATATATTCAATGGGCCAGCGCAGAGCAGACATTAGT | 38 |
| | TTTTCATCGTGGTGGTTATTGT | |
| P72-F2-GGCC | CATATATTCAATGGGCCAGGCCAGAGCAGACATTAGT | 39 |
| | TTTTCATCGTGGTGGTTATTGT | |
| P72-F2-GGGC | CATATATTCAATGGGCCAGGGCAGAGCAGACATTAGT | 40 |
| | TTTTCATCGTGGTGGTTATTGT | |

In this embodiment, the selected different PAM target double-stranded DNA (dsDNA) is the African swine fever P72 gene, its sequence: CTGTAACGCAGCACAGCTGAACCGTTCTGAAGAAGAAGAAAGTTAATAGCAGATGCC GATACCACAAGATCAGCCGTAGTGATAGACCCCACGTAATCCGTGTCCCAACTAATAT AAAATTCTCTTGCTCTGGATACGTTAATATGACCACTGGGTTGGTATTCCTCCCGTGGC TTCAAAGCAAAGGTAATCATCATCGCACCCGGATCATCGGGGGTTTTAATCGCATTGC CTCCGTAGTGGAAGGGTATGTAAGAGCTGCAGAACTTTGATGGAAATTTATCGATAAG ATTGATACCATGAGCAGTTACGGAAATGTITTTAATAATAGGTAATGTGATCGGATACG TAACGGGGCTAATATCAGATATAGATGAACATGCGTCTGGAAGAGCTGTATCTCTATCC TGAAAGCTTATCTCTGCGTGGTGAGTGGGCTGCATAATGGCGTTAACAACATGTCCGA ACTTGTGCCAATCTCGGTGTTGATGAGGATTTTGATCGGAGATGTTCCAGGTAGGTTT TAATCCTATAAACATATATTCAATGGGCCANNNNAGAGCAGACATTAGTTTTTCATCGT GGTGGTTATTGTTGGTGTGGGTCACCTGCGTTTTATGGACACGTATCAGCGAAAAGCG AACGCGTTTTACAAAAAGGTTGTGTATTTCAGGGGTTACAAACAGGTTATTGATGTAA AGTTCATTATTCGTGAGCGAGATTTCATTAATGACTCCTGGGATAAACCATGG (SEQ ID NO: 41); the bold marking is PAM, and the underline represents the target sequence. For the same guide RNA: AAUUUCUACUAUUGUAGAUUAGAGCAGACAUUAGUUUUUC (SEQ ID NO: 42) (the underlined area is the target area).

Secondly, the DNA sequence encoding Gs12-10 was synthesized by optimizing the Escherichia coli codon, and NLS nuclear localization signals were added to its C-terminus. The DNA sequence is shown in SEQ ID NO: 3. Subsequently, it was connected to the prokaryotic expression vector pET-28a and transformed into the Escherichia coli BL21 strain. After identifying the positive clone, IPTG-induced expression was performed, and the target protein was purified through affinity chromatography. The following system was adopted in the in vitro cleaving reaction: 10×CutSmart Buffer 2 μL. The predicted Gs12-10-NLS-tagged protein was 500 ng, the guide RNA was 500 ng, and the P72 target PCR amplification product for different PAMs was 2 μL. The system was incubated at 37° C. for 30 min, respectively. After the reaction was completed, 1 μL of protease K was added separately and incubated at 55° C. for 10 min to terminate the reaction. Guide RNA and target nucleic acid were added in the experimental group, while guide RNA was not added in the control group. After the reaction, 1% agarose gel electrophoresis was carried out, and the difference between the target bands of the new endonuclease Gs12-10 predicted under different PAM target sites in the experimental group and the control group was detected by imaging observation under UV transilluminator, and the cleaving efficiency was analyzed through Image J software.

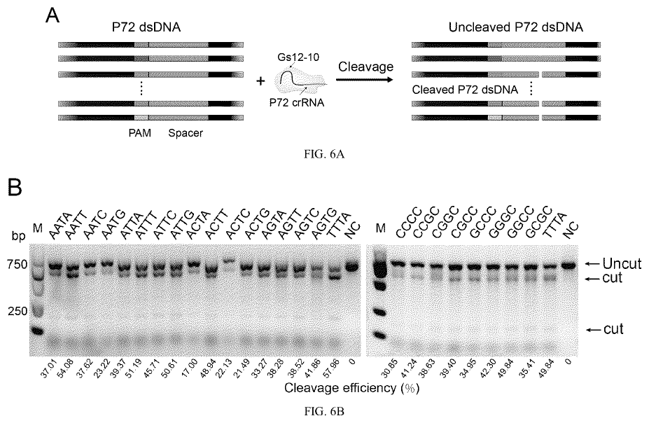

As shown in FIGS. 6A-6B, target sites targeting the same crRNA as the P72 gene but with different PAM sequences are randomly selected for experimental verification. Similar to the Cas12a mutant with a known PAM sequence of “TNTN”, the Gs12-10 protein can cleave the target site with “ANTN” as PAM. In addition, the Gs12-10 protein can recognize PAM sequences as target sites containing cytosine and guanine but not thymidine nucleosides, such as “CCCC”, “CCGC”, “GGGC”, etc., indicating its high cleavage activity towards non-classical PAMs. From this, it can be seen that the Gs12-10 protein found through the bacterial PAM library subtraction experiment is reliable without PAM restriction.

To further confirm the reliability of the conclusion without PAM restriction, we validated it by in vitro enzyme digestion of circular plasmids. CrRNAs with different PAM sequences for the puc19 vector were randomly designed, and multiple different guide RNAs (crRNAs) were designed for the PAM site of “ANTN”. crRNA-AATA, crRNA-AATT, crRNA-AATC, crRNA-AATG, crRNA-ATTA, crRNA-ATTT, crRNA-ATTC, crRNA-ATTG, crRNA-ACTA, crRNA-ACTT, crRNA-ACTC, crRNA-ACTG, crRNA-AGTA, crRNA-AGTT, crRNA-AGTC and crRNA-AGTG. crRNA sequences are:

(SEQ ID NO: 43)
AAUUUCUACUAUUGUAGAUUCGGTTATCCACAGAATCAGG,
(SEQ ID NO: 44)
AAUUUCUACUAUUGUAGAUUAATGTGAGTTAGCTCACTCA,
(SEQ ID NO: 45)
AAUUUCUACUAUUGUAGAUUTGCTCTGATGCCGCATAGTT,
(SEQ ID NO: 46)
AAUUUCUACUAUUGUAGAUUAATCGGCCAACGCGCGGGGA,
(SEQ ID NO: 47)
AAUUUCUACUAUUGUAGAUUAGCGGGCAGTGAGCGCAACG,
(SEQ ID NO: 48)
AAUUUCUACUAUUGUAGAUUCACACAGGAAACAGCTATGA,
(SEQ ID NO: 49)
AAUUUCUACUAUUGUAGAUUATTAATGCAGCTGGCACGAC,
(SEQ ID NO: 50)
AAUUUCUACUAUUGUAGAUUGGCGCTCTTCCGCTTCCTCG,
(SEQ ID NO: 51)
AAUUUCUACUAUUGUAGAUUTTTCTCAGAATGACTTGGTTG,
(SEQ ID NO: 52)
AAUUUCUACUAUUGUAGAUUGAGCGTCGATTTTTGTGATG,
(SEQ ID NO: 53)
AAUUUCUACUAUUGUAGAUUTAGAGGATCCCCGGGTACCG,
(SEQ ID NO: 54)
AAUUUCUACUAUUGUAGAUUACTCGCTGCGCTCGGTCGTT,
(SEQ ID NO: 55)
AAUUUCUACUAUUGUAGAUUTGAGTATTCAACATTTCCGT,
(SEQ ID NO: 56)
AAUUUCUACUAUUGUAGAUUGGGTAACGCCAGGGTTTTCC,
(SEQ ID NO: 57)
AAUUUCUACUAUUGUAGAUUAGAGGTGGCGAAACCCGACA,
(SEQ ID NO: 58)
AAUUUCUACUAUUGUAGAUUAGCTGATACCGCTCGCCGCA.

The underline represents the target sequence. The following system was adopted in the in vitro cleaving reaction: 10×CutSmart Buffer 2 μL. The predicted Gs12-10-NLS-tagged protein was 500 ng, the guide RNA was 500 ng, and the puc19 vector was 200 ng. The system was incubated at 37° C. for 2 h, respectively. After the reaction was completed, 1 μL of protease K was added separately and incubated at 55° C. for 10 min to terminate the reaction. Guide RNA and target nucleic acid were added in the experimental group, while guide RNA was not added in the control group. After the reaction, 1% agarose gel electrophoresis was carried out, and the difference between the target bands of the new endonuclease Gs12-10 predicted under different PAM target sites in the experimental group and the control group was detected by imaging observation under UV transilluminator, and the cleaving efficiency was analyzed through Image J software.

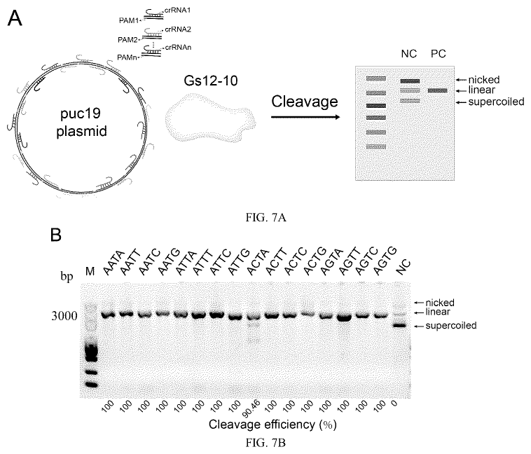

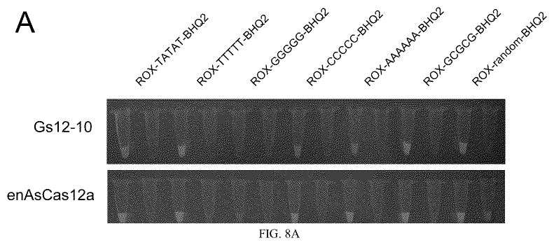

As shown in FIGS. 7A-7B, for different crRNAs of puc19 plasmids at different PAM sites, Gs12-10 can cleave plasmid DNA of different PAMs in the reaction solution at the target site with “ANTN” as PAM, and can cut all plasmids with a superhelical structure into linear structures with 100% efficiency. Although not all cleavage occurred at the target site with “ACTA” as PAM, it may be due to the activity of crRNA itself. This further proves the reliability of the identification results of the bacterial PAM library subtraction experiment.

### Example 4. Establishment of an On-Site Visualization and Rapid Detection Platform for Nucleic Acids Using the CRISPR-Gs12-10 System

Further, evaluate whether the Gs12-10 protein has trans-cleavage activity. Guide RNA that could pair with the target nucleic acid was used to guide endonuclease Gs12-10 to recognize and bind to the target nucleic acid; subsequently, its “trans-cleavage” activity towards any single-stranded nucleic acid was stimulated, thereby cleaving the single-stranded DNA fluorescence quenching reporter gene (ssDNA-FQ) in the reaction system; furthermore, the trans-cleavage function of the candidate bacterial protein could be determined by the excitation fluorescence intensity, background noise, and visual color changes. By screening different base combinations of intermediate single-stranded DNA, the optimal fluorescence quenching reporter gene (ssDNA-FQ) for the Gs12-10 protein was identified.

The target double-stranded DNA (dsDNA) used in this embodiment is the p72 conserved gene of African swine fever virus ASFV, its sequence: CTGTAACGCAGCACAGCTGAACCGTTCTGAAGAAGAAGAAAGTTAATAGCAGATGCC GATACCACAAGATCAGCCGTAGTGATAGACCCCACGTAATCCGTGTCCCAACTAATAT AAAATTCTCTTGCTCTGGATACGTTAATATGACCACTGGGTTGGTATTCCTCCCGTGGC TTCAAAGCAAAGGTAATCATCATCGCACCCGGATCATCGGGGGTTTTAATCGCATTGC CTCCGTAGTGGAAGGGTATGTAAGAGCTGCAGAACTTTGATGGAAATTATCGATAAG ATTGATACCATGAGCAGTTACGGAAATGTITTTAATAATAGGTAATGTGATCGGATACG TAACGGGGCTAATATCAGATATAGATGAACATGCGTCTGGAAGAGCTGTATCTCTATCC TGAAAGCTTATCTCTGCGTGGTGAGTGGGCTGCATAATGGCGTTAACAACATGTCCGA ACTTGTGCCAATCTCGGTGTTGATGAGGATTTTGATCGGAGATGTTCCAGGTAGGTTT TAATCCTATAAACATATATTCAATGGGCCATTTAAGAGCAGACATTAGTTTTTCATCGT GGTGGTTATTGTTGGTGTGGGTCACCTGCGTTTTATGGACACGTATCAGCGAAAAGCG AACGCGTTTTACAAAAAGGTTGTGTATTTCAGGGGTTACAAACAGGTTATTGATGTAA AGTTCATTATTCGTGAGCGAGATTTCATTAATGACTCCTGGGATAAACCATGG (SEQ ID NO: 5); the bold marking is PAM, and the underline represents the target sequence. The guide RNA sequence is: AAUUUCUACUAUUGUAGAUUAGAGCAGACAUUAGUUUUUC (SEQ ID NO: 42) (the underlined area is the target area). The single-stranded DNA fluorescence quenching reporter gene sequences are ROX-TATAT-BHQ2, ROX-ITITTT-BHQ2, ROX-GGGGG-BHQ2, ROX-CCCCC-BHQ2, ROX-AAAAA-BHQ2, ROX-GCGCG-BHQ2, or ROX-random-BHQ 2 (5′ROX/GTATCCAGTGCG/3′BHQ2) (SEQ ID NO: 134). Firstly, Gs12-10 and the known enhanced Cas12a protein were purified by prokaryotic expression, followed by in vitro transcription of guide RNA and PCR amplification of p72 target gene double-stranded DNA. Next, the following reaction system was adopted: Gs12-10 or enAsCas12a protein 500 ng, guide RNA 500 ng, 2 μL of 10×CutSmartBuffer, 1 μM of single-stranded DNA fluorescence quenching reporter genes with different base combinations, and 2 μL of PCR amplification target product. The negative control was without a target. The reaction was carried at 37° C. for 15 min, and 98° C. for 2 min to inactivate. By observing fluorescence intensity and background noise quantitatively on a microplate reader and under blue light, the preferred probe for predicting the trans-cleavage activity of the above proteins in vitro could be determined.

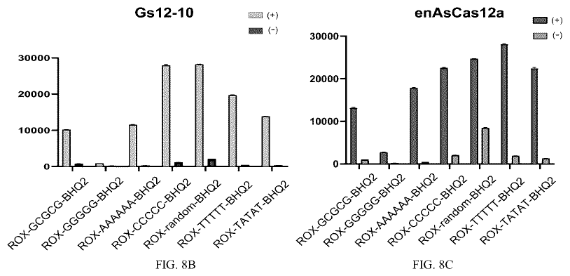

As shown in FIGS. 8A-8C, from the fluorescence changes of the reaction solution before and after cleavage, it can be seen that the newly discovered Gs12-10 protein and the known enhanced enAsCas12a protein both have nucleic acid trans-cleavage activity; compared with the known enAsCas12a, the activated newly identified protein can not only transcleave ROX-GCGCG-BHQ2 and ROX-random-BHQ2, but also cleave ROX-TATAT-BHQ2, ROX-TTTTT-BHQ2, ROX-CCCCC-BHQ2, and ROX-AAAAA-BHQ2 probes. Therefore, the newly discovered Gs12-10 protein has a wide range of probe types for trans-cleavage. Compared with the enhanced enAsCas12a, it is found that Gs12-10 has relatively higher trans-cleavage activity and lower non-specific cleavage activity.

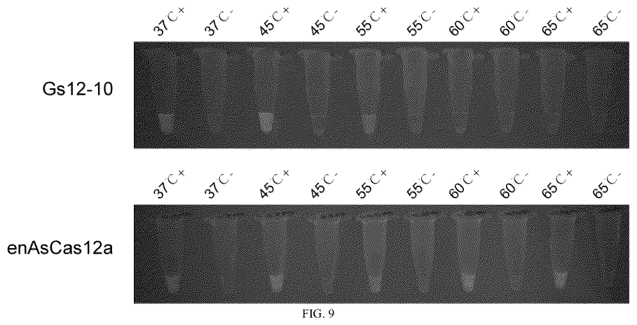

Subsequently, the optimal enzyme digestion reaction temperature for the nucleic acid detection technology mediated by the Gs12-10 system was evaluated. Using the above targets as the nucleic acid detection sites, the following system reactions were performed: Gs12-10 protein 500 ng, guide RNA 500 ng, 2 μL of 10×CutSmart Buffer, 1 μM of single-stranded DNA fluorescence quenching reporter genes (ROX-random-BHQ2), and 2 μL of P72 PCR amplification target product. The negative control was without a target. The reaction was carried at 37° C., 45° C., 55° C., 60° C., and 65° C. for 15 min separately, and 98° C. for 2 min to inactivate. Fluorescence intensity and background noise were observed under blue light. As shown in FIG. 9, the optimal reaction temperature for Gs12-10 endonuclease cleavage is 37° C.-55° C. Compared with the known enhanced enAsCas12a, although its temperature tolerance range is lower, its background noise is relatively lower.

Finally, to verify whether the Gs12-10 protein can also be used for nucleic acid detection without being restricted by PAM, crRNA was designed for the PAM site of the above puc19 vector “ANTN” for detection. Using the following system reactions: Gs12-10/LbCas12a protein 500 ng, guide RNA 500 ng for different PAMs, 2 μL of 10×CutSmart Buffer, 1 μM of single-stranded DNA fluorescence quenching reporter gene (ROX-random-BHQ2) and 200 ng of puc19 vector. The negative control was a vector without a target. The reaction was carried at 37° C. for 15 min, and 98° C. for 2 min to inactivate. Fluorescence intensity and background noise were observed under blue light. Secondly, for the same crRNA of above P72, different PAM target P72 genes amplified by overlay PCR were validated for nucleic acid detection using the above reaction system and conditions. Finally, different crRNAs were designed for different PAM target sites of the P72 gene, including crRNA, crRNA-ATTV-3, crRNA-TTTV-1, crRNA-TTTV2, crRNA-TTTV-3, crRNA-CTTV-1, crRNA-CTTV-2, crRNA-CTTV-3, crRNA-GTTV-1, crRNA-GTTV-2, crRNA-GTTV-3, crRNA-PC and crRNA-AAAA, crRNA-AAAT, crRNA-AAAC, crRNA-AAAG, crRNA-GGGA, crRNA-GGGT, crRNA-GGGC, crRNA-GGGG, crRNA-CCCA, crRNA-CCCT, crRNA-CCCG, crRNA-CCCC, etc., which is as shown in the table below.

| | | SEQ ID |
|---|---|---|
| crRNA name | Sequence (5′-3′) | NO: |
| crRNA-ATTV-3 | AAUUUCUACUAUUGUAGAUUAUACCAUGAGCAGUU | 59 |
| | ACGGA | |
| crRNA-TTTV-1 | AAUUUCUACUAUUGUAGAUUAAGCCACGGGAGGAA | 60 |
| | UACCA | |
| crRNA-TTTV-2 | AAUUUCUACUAUUGUAGAUUCACUACGGAGGCAAUG | 61 |
| | CGAU | |
| crRNA-TTTV-3 | AAUUUCUACUAUUGUAGAUUCGUAACUGCUCAUGGU | 62 |
| | AUCA | |
| crRNA-CTTV-1 | AAUUUCUACUAUUGUAGAUUAAAGCAAAGGUAAUC | 63 |
| | AUCAU | |
| crRNA-CTTV-2 | AAUUUCUACUAUUGUAGAUUGAUGGAAAUUUAUCG | 64 |
| | AUAAG | |
| crRNA-CTTV-3 | AAUUUCUACUAUUGUAGAUUCAUACCCUUCCACUAC | 65 |
| | GGAG | |
| crRNA-GTTV-1 | AAUUUCUACUAUUGUAGAUUCGGAAAUGUUUUUAA | 66 |
| | UAAUA | |
| crRNA-GTTV-2 | AAUUUCUACUAUUGUAGAUUAUCUAUAUCUGAUAU | 67 |
| | UAGCC | |
| crRNA-GTTV-3 | AAUUUCUACUAUUGUAGAUUUUAAUAAUAGGUAAU | 68 |
| | GUGAU | |
| crRNA-PC | AAUUUCUACUAUUGUAGAUUAGAGCAGACAUUAGU | 42 |
| | UUUUC | |
| crRNA-AAAG | AAUUUCUACUAUUGUAGAUUCAAAGGUAAUCAUCA | 69 |
| | UCGCA | |
| crRNA-AAAT | AAUUUCUACUAUUGUAGAUUUUAUCGAUAAGAUUG | 70 |
| | AUACC | |
| crRNA-AAAC | AAUUUCUACUAUUGUAGAUUAUAUAUUCAAUGGGC | 71 |
| | CAUUU | |
| crRNA-AAAA | AAUUUCUACUAUUGUAGAUUUCCUCAUCAACACCGA | 72 |
| | GAUU | |
| crRNA-CCCC | AAUUUCUACUAUUGUAGAUUACGUAAUCCGUGUCCC | 73 |
| | AACU | |
| crRNA-CCCG | AAUUUCUACUAUUGUAGAUUUGGCUUCAAAGCAAA | 74 |
| | GGUAA | |
| crRNA-CCCA | AAUUUCUACUAUUGUAGAUUACUAAUAUAAAAUUC | 75 |
| | UCUUG | |
| crRNA-CCCT | AAUUUCUACUAUUGUAGAUUUCCACUACGGAGGCAA | 76 |
| | UGCG | |
| crRNA-GGGC | AAUUUCUACUAUUGUAGAUUUGCAUAAUGGCGUUA | 77 |
| | ACAAC | |
| crRNA-GGGA | AAUUUCUACUAUUGUAGAUUCACGGAUUACGUGGG | 78 |
| | GUCUA | |
| crRNA-GGGT | AAUUUCUACUAUUGUAGAUUUGGUAUUCCUCCCGUG | 79 |
| | GCUU | |
| crRNA-GGGG | AAUUUCUACUAUUGUAGAUUGUUACGUAUCCGAUCA | 80 |
| | CAUU | |
| crRNA-AAGA | AAUUUCUACUAUUGUAGAUUUCAGCCGUAGUGAUA | 81 |
| | GACCC | |
| crRNA-AAGC | AAUUUCUACUAUUGUAGAUUAAAGGUAAUCAUCAU | 82 |
| | CGCAC | |
| crRNA-AAGG | AAUUUCUACUAUUGUAGAUUGUAUGUAAGAGCUGC | 83 |
| | AGAAC | |
| crRNA-AACT | AAUUUCUACUAUUGUAGAUUAAUAUAAAAUUCUCU | 84 |
| | UGCUC | |
| crRNA-AACC | AAUUUCUACUAUUGUAGAUUCAGUGGUCAUAUUAA | 85 |
| | CGUAU | |
| crRNA-AACG | AAUUUCUACUAUUGUAGAUUGGGCUAAUAUCAGAU | 86 |
| | AUAGA | |
| crRNA-AGAG | AAUUUCUACUAUUGUAGAUUCUGUAUCUCUAUCCUG | 87 |
| | AAAG | |
| crRNA-AGAT | AAUUUCUACUAUUGUAGAUUGUUCCAGGUAGGUUU | 88 |
| | UAAUC | |
| crRNA-AGAA | AAUUUCUACUAUUGUAGAUUCUUUGAUGGAAAUUU | 89 |
| | AUCGA | |
| crRNA-AGGG | AAUUUCUACUAUUGUAGAUUUAUGUAAGAGCUGCA | 90 |
| | GAACU | |
| crRNA-AGGT | AAUUUCUACUAUUGUAGAUUAAUGUGAUCGGAUAC | 91 |
| | GUAAC | |
| crRNA-AGGA | AAUUUCUACUAUUGUAGAUUUAGAGAUACAGCUCU | 92 |
| | UCCAG | |
| crRNA-AGCA | AAUUUCUACUAUUGUAGAUUGAUGCCGAUACCACAA | 93 |
| | GAUC | |
| crRNA-AGCC | AAUUUCUACUAUUGUAGAUUACGGGAGGAAUACCA | 94 |
| | ACCCA | |
| crRNA-AGCT | AAUUUCUACUAUUGUAGAUUGCAGAACUUUGAUGG | 95 |
| | AAAUU | |
| crRNA-ACAC | AAUUUCUACUAUUGUAGAUUGGAUUACGUGGGGUC | 96 |
| | UAUCA | |
| crRNA-ACAT | AAUUUCUACUAUUGUAGAUUACCCUUCCACUACGGA | 97 |
| | GGCA | |
| crRNA-ACAG | AAUUUCUACUAUUGUAGAUUCUCUUCCAGACGCAUG | 98 |
| | UUCA | |
| crRNA-ACGT | AAUUUCUACUAUUGUAGAUUUAAUAUGACCACUGG | 99 |
| | GUUGG | |
| crRNA-ACGG | AAUUUCUACUAUUGUAGAUUAGGCAAUGCGAUUAA | 100 |
| | AACCC | |
| crRNA-ACGC | AAUUUCUACUAUUGUAGAUUAUGUUCAUCUAUAUC | 101 |
| | UGAUA | |
| crRNA-ACCC | AAUUUCUACUAUUGUAGAUUGGAUCAUCGGGGGUU | 102 |
| | UUAAU | |
| crRNA-ACCA | AAUUUCUACUAUUGUAGAUUUGAGCAGUUACGGAA | 103 |
| | AUGUU | |
| crRNA-ACCT | AAUUUCUACUAUUGUAGAUUAUUAUUAAAAACAUU | 104 |
| | UCCGU | |

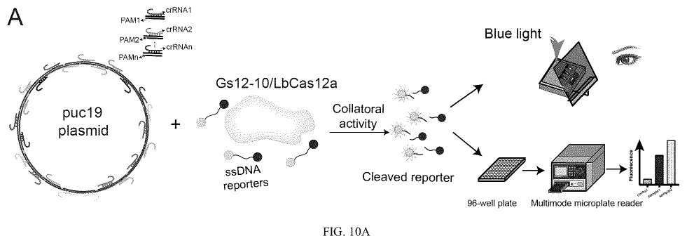

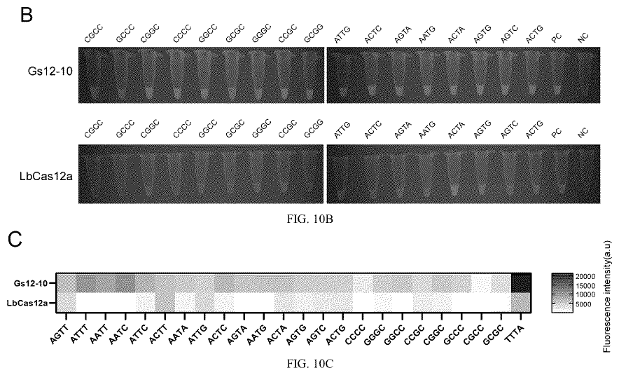

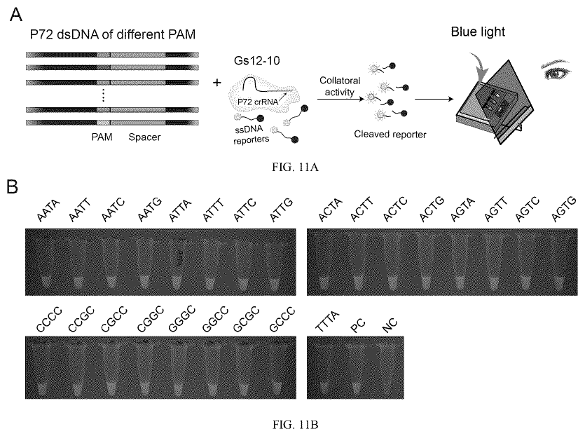

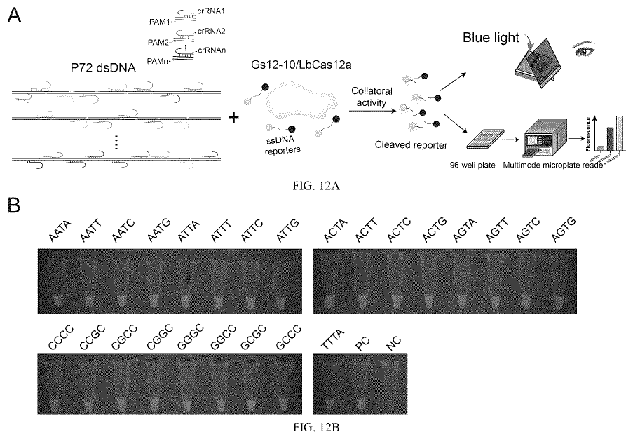

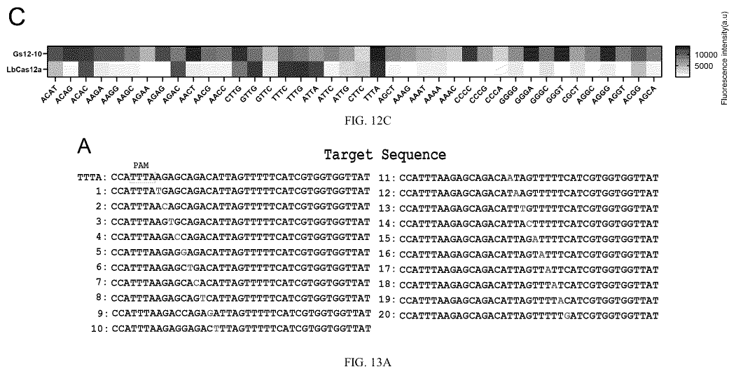

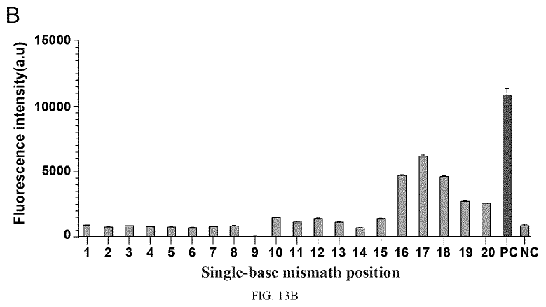

As shown in FIGS. 10A-10C, compared to the known LbCas12a, the nucleic acid detection of puc19 vector by the Gs12-10 protein is indeed not limited by PAM, such as “ATTT”, “AATC”, “AATG”, and “GGCC”. Meanwhile, in nucleic acid detection targeting different PAM P72 genes for the same ASFV-P72 crRNA, the Gs12-10 protein shows high trans-cleavage activity, with all targets and positive control targets exhibiting high activity (FIGS. 11A-11B). Finally, when conducting nucleic acid detection on different crRNAs and non-classical PAM targets of the P72 gene, Gs12-10 still exhibits high nucleic acid detection activity, such as “ANTN”, “CCCB”, “GGGN”, “AAAH” and other PAM sites (N=A/T/G/C, B=G/T/C, and H=A/T/C). However, known LbCas12a exhibits high nucleic acid detection activity similar to that of Gs12-10 only at classical PAM sites such as “TTTV”. It is worth mentioning that LbCas12a also exhibits good activity in some non-classical PAMs such as “ACAC” and “AGAC” (FIGS. 12A-12C). Overall, Gs12-10 has high detection activity for different target sites in this test, such as plasmid DNA and PCR amplification target fragments for different PAMs. Therefore, the nucleic acid visualization detection mediated by Gs12-10 protein is not restricted by PAM and has a wider range of target recognition.

### Example 5. Assessing the Positional Impact of Single Base Mismatches on Gs12-10 Cis-Cleavage Activity at Target Sites

Further identification of the CRISPR-Gs12-10 system's ability to recognize single base mismatches in the non-PAM region. The target double-stranded DNA (dsDNA) used in this embodiment is the p72 conserved gene of African swine fever virus ASFV, its sequence: CCATTTAAGAGCAGACATTAGTTTTTCATCGTGGTGGTTATTGTTGGTGTGGGTCACC TGCGTTTTATGGACACGTATCAGCGAAAAGCGAACGCGTTTTACAAAAAGGTTGTGTA TTTCAGGGGTTACAAACAGGTTATT (SEQ ID NO: 105), the bold marking is PAM, and the underline represents the target sequence. Firstly, PCR amplification was performed to obtain a double-stranded DNA template containing mutations from the 1-20 consecutive target sites. Target-F to Target-p72-F-20G primers were used upstream, and Target-p72-R primers were used downstream to amplify to obtain the target double-stranded gene. The primer sequence table used in the present invention is as follows:

| | | SEQ ID |
|---|---|---|
| Primer name | Sequence (5′-3′) | NO: |
| Target-F | CCATTTAAGAGCAGACATTAGTTTTTCA | 106 |
| Target-p72-F-1T | CCATTTATGAGCAGACATTAGTTTTTCA | 107 |
| Target-p72-F-2C | CCATTTAACAGCAGACATTAGTTTTTCA | 108 |
| Target-p72-F-3T | CCATTTAAGTGCAGACATTAGTTTTTCA | 109 |
| Target-p72-F-4C | CCATTTAAGACCAGACATTAGTTTTTCA | 110 |
| Target-p72-F-5G | CCATTTAAGAGGAGACATTAGTTTTTCA | 111 |
| Target-p72-F-6T | CCATTTAAGAGCTGACATTAGTTTTTCA | 112 |
| Target-p72-F-7C | CCATTTAAGAGCACACATTAGTTTTTCA | 113 |
| Target-p72-F-8T | CCATTTAAGAGCAGTCATTAGTTTTTCA | 114 |
| Target-p72-F-9G | CCATTTAAGAGCAGAGATTAGTTTTTCA | 115 |
| Target-p72-F-10T | CCATTTAAGAGCAGACTTTAGTTTTTCA | 116 |
| Target-p72-F-11A | CCATTTAAGAGCAGACAATAGTTTTTCATCGTGGTG | 117 |
| Target-p72-F-12A | CCATTTAAGAGCAGACATAAGTTTTTCATCGTGGTG | 118 |
| Target-p72-F-13T | CCATTTAAGAGCAGACATTTGTTTTTCATCGTGGTG | 119 |
| Target-p72-F-14C | CCATTTAAGAGCAGACATTACTTTTTCATCGTGGTG | 120 |
| Target-p72-F-15A | CCATTTAAGAGCAGACATTAGATTTTCATCGTGGTG | 121 |
| Target-p72-F-16A | CCATTTAAGAGCAGACATTAGTATTTCATCGTGGTG | 122 |
| Target-p72-F-17A | CCATTTAAGAGCAGACATTAGTTATTCATCGTGGTG | 123 |
| Target-p72-F-18A | CCATTTAAGAGCAGACATTAGTTTATCATCGTGGTG | 124 |
| Target-p72-F-19A | CCATTTAAGAGCAGACATTAGTTTTACATCGTGGTG | 125 |
| Target-p72-F-20G | CCATTTAAGAGCAGACATTAGTTTTTGATCGTGGTG | 126 |
| Target-p72-R | CAATAACCTGTTTGTAACCCCTGAAATAC | 127 |

Among them, the guide RNA sequence is: AAUUUCUACUAUUGUAGAUUAGAGCAGACAUUAGUUUUUC (SEQ ID NO: 42) (the underlined area is the target area). The single-stranded DNA fluorescence quenching reporter gene sequence is ROX-random-BHQ2 (5′ROX/GTATCCAGTGCG/3′BHQ2); firstly, the Gs12-10 protein was obtained by prokaryotic expression purification, guide RNA was obtained by in vitro transcription, and the target gene DNA with p72 single base mutation was obtained by PCR amplification. Next, the following reaction system was adopted: Gs12-10 protein 500 ng, guide RNA 500 ng, 2 μL of 10×CutSmart NE Buffer, 1 μM of single-stranded DNA fluorescence quenching reporter (ROX-random-BHQ2), and 2 μL of PCR amplification target products with different base mutations. By accurately reading fluorescence intensity and background noise on a microplate reader, the single base mismatch recognition ability of the Gs12-10 protein was determined, and its recognition specificity was evaluated accordingly.

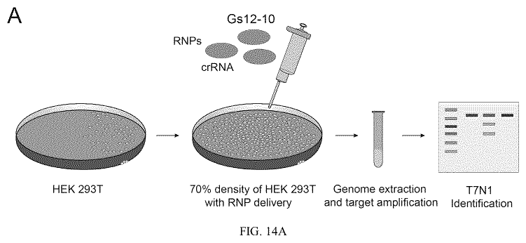

As shown in FIGS. 13A-13B, compared with a completely paired positive control, the presence of many single base mismatch sites can significantly inhibit the dsDNA cleavage activity of the Gs12-10 protein, and the activity is almost consistent with negative, such as single base mismatch in positions 1-15. From this, it can be seen that the Gs12-10 protein is highly sensitive to single base mismatches at target sites, which in turn indicates its high specificity for target site recognition and is more helpful for precise detection of single nucleotide sequence polymorphism (SNP) or base editing techniques in the future.

### Example 6. Development of a CRISPR-Gs12-10 System-Mediated Genome Editing Platform

The ability of the Gs12-10 protein to target and cleave genomes was evaluated in this embodiment. Specifically, newly discovered Gs12-10 and enAsCas12a proteins were first incubated with guide RNA using Lipofectamine™ CRISPRMAX™, after which their activities were compared. Subsequently, ribonucleoprotein complexes (RNPs) formed by each protein were transfected into human HEK293T cells. The guide RNA, paired with the target DNA, guided the recognition and binding of Gs12-10 and enAsCas12a proteins to the target DNA, thereby enabling genome cleavage. Finally, cells were collected, genomic DNA was extracted, and the cleavage activity was detected through T7EN1 digestion and agarose gel electrophoresis.

In this embodiment, the selected target nucleic acid is the human FANCF gene, PAM is TTTG, its sequence: GCCCTACATCTGCTCTCCCTCCACTAAGAAGAACCTCTTTGTGTGGCGAAAGTAAAA GTATTAGGGCTTTTAAGTTGCCCAGAGTCAAGGAACACGGATAAAGACGCTGGGAGA TTGACATGCATTTCGACCAATAGCATTGCAGAGAGGCGTATCATTTCGCGGATGTTCC AATCAGTACGCAGAGAGTCGCCGTCTCCAAGGTGAAAGCGGAAGTAGGGCCTTCGC GCACCTCATGGAATCCCTTCTGCAGCACCTGGATCGCTTTTCCGAGCTTCTGGCGGTC TCAAGCACTACCTACGTCAGCACCTGGGACCCCGCCACCGTGCGCCGGGCCTTGCAG TGGGCGCGCTACCTGCGCCACATCCATCGGCGCTTTGGTCGGCATGGCCCCATTCGC ACGGCTCTGGAGCGGCGGCTGCACAACCAGTGGAGGCAAGAGGGCGGCTTTGGGCG GGGTCCAGTTCCGGGATTAGCGAACTTCCAGGCCCTCGGTCACTGTGACGTCCTGCT CTCTCTGCGCCTGCTGGAGAACCGGGCCCTCGGGGATGCAGCTCGTTACCACCTGGT GCAGCAACTCTTTCCCGGCCC (SEQ ID NO: 128); the bold part is the PAM sequence, the underlined area is the target area. The guide RNA sequence is: AAUUUCUACUAUUGUAGAUUGUCGGCAUGGCCCCAUUCGC (SEQ ID NO: 129) (the underlined area is the target area); when the fusion degree of HEK 293T cells reached 70/6-80%, they were planked and inoculated into a 12-well plate at 8×10⁴ cells/well. Transfection was carried out after 6-8 hours of planking, and 1.25 μg of the predicted Gs12-10 or enAsCas12a-NLS-tagged protein was added and incubated with 625 ng of guide RNA, mixed well with 50 μL of Lopti-MEM and 2.6 μL of Cas9 Plus™ reagent; 50 μL of opti-MEM was mixed well with 3 μL of CRISPR™ reagent. Diluted CRISPR™ reagent was mixed well with diluted RNP and incubated at room temperature for 10 min. The incubated mixture was added to the culture medium covered with cells for transfection. After incubating at 37° C. for 72 hours, the culture medium was discarded and 100 μL of PBS was used to perform cell resuspension to extract the genome of cells. PCR amplification was performed on the target sites of transfected positive cells. T7EN1 enzyme treatment reaction and agarose gel electrophoresis were used to observe the changes of bands to judge whether the predicted protein had gene editing activity in vivo, and Image J was used to roughly calculate the editing efficiency. The template for the negative control consists of genomic DNA from normally cultured HEK 293T cells that have not been transfected with RNP.

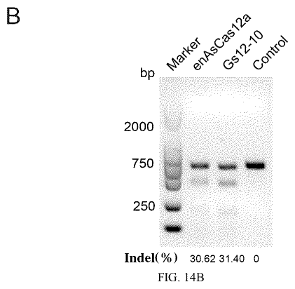

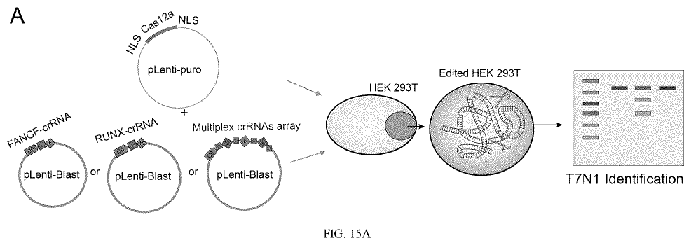

As illustrated in FIGS. 14A-14B, the enAsCas12a and Gs12-10 proteins in the experimental group exhibit significant genome editing activity, as detected by T7EN1 digestion reaction and electrophoresis, compared to the negative control without RNP transfection. Their cleaving efficiencies (Indel) are 30.62% and 31.40%, respectively. These results indicate that the newly discovered Gs12-10 protein is capable of inducing RNA-guided genome editing in human cells.

Further, in this embodiment, the eukaryotic cell codon of the newly discovered Gs12-10 protein was optimized, and SV40 NLS and NLS nuclear localization signals were added to the N and C terminals of its protein, respectively. The sequence was shown in SEQ ID NO: 4. The synthesized sequence was constructed into Lenti-puro lentivirus vector, and at the same time, it was co-transfected with the guide RNA eukaryotic expression vector to HEK293T cells through liposomes. A guide RNA paired with the target DNA was used to direct the Gs12-10 protein to recognize and cleave the target DNA molecule, and its RNA-guided genome editing activity was detected by T7EN1 digestion followed by agarose gel electrophoresis.

The selected target nucleic acid is the human FANCF gene, PAM is TTTG, its sequence: GCCCTACATCTGCTCTCCCTCCACTAAGAAGAACCTCTTTGTGTGGCGAAAGTAAAA GTATTAGGGCTTTTAAGTTGCCCAGAGTCAAGGAACACGGATAAAGACGCTGGGAGA TTGACATGCATTTCGACCAATAGCATTGCAGAGAGGCGTATCATTTCGCGGATGTTCC AATCAGTACGCAGAGAGTCGCCGTCTCCAAGGTGAAAGCGGAAGTAGGGCCTTCGC GCACCTCATGGAATCCCTTCTGCAGCACCTGGATCGCTTTTCCGAGCTTCTGGCGGTC TCAAGCACTACCTACGTCAGCACCTGGGACCCCGCCACCGTGCGCCGGGCCTTGCAG TGGGCGCGCTACCTGCGCCACATCCATCGGCGCTTTGGTCGGCATGGCCCCATTCGC ACGGCTCTGGAGCGGCGGCTGCACAACCAGTGGAGGCAAGAGGGCGGCTTTGGGCG GGGTCCAGTTCCGGGATTAGCGAACTTCCAGGCCCTCGGTCACTGTGACGTCCTGCT CTCTCTGCGCCTGCTGGAGAACCGGGCCCTCGGGGATGCAGCTCGTTACCACCTGGT GCAGCAACTCTTTCCCGGCCC (SEQ ID NO: 128); the bold part is the PAM sequence, the underlined area is the target area. The guide RNA sequence is: AAUUUCUACUAUUGUAGAUUGUCGGCAUGGCCCCAUUCGC (SEQ ID NO: 129) (the underlined area is the target area); and the human RUNX1 gene, PAM is TTTC, its sequence: CATCACCAACCCACAGCCAAGGCGGCGCTGGCTTTTTTTTTTTTTTTAATCTTTAACAA TTTGAATATTTGTTTTTACAAAGGTGCATTTTTTAATAGGGCTTGGGGAGTCCCAGAGG TATCCAGCAGAGGGGAGAAGAAAGAGAGATGTAGGGCTAGAGGGGTGAGGCTGAAA CAGTGACCTGTCTTGGTTTTCGCTCCGAAGGTAAAAGAAATCATTGAGTCCCCCGCC TTCAGAAGAGGGTGCATTTTCAGGAGGAAGCGATGGCTTCAGACAGCATATTTGAGT CATTTCCTTCGTACCCACAGTGCTTCATGAGAGGTGAGTACATGCTGGTCTTGTAATAT CTACTTTGCTCAGCTTTGCCTGTAATGAAATGGCAGCTTGTTTCACCTCGGTGCAGA GATGCCTCGGTGCCTGCCAGTTCCCTGTCTTGTTTGTGAGAGGAATTCAAACTGAGGC ATATGATTACAAGTCTATTGGATTACTTACTAATCAGATGGAAGCTCTTCAGAAATGTT TTAATAAATACTTAGTTATGCTGTTGGAGTGTTCAGTCGGTGCGTGAGAACTTTGTCA AGTGCGAGTAAGTTGTGCTGG (SEQ ID NO: 130), the bold part is the PAM sequence, the underlined area is the target area. The human EMX1 gene, PAM is TTTG, its sequence: GGAGCAGCTGGTCAGAGGGGACCCCGGCCTGGGGCCCCTAACCCTATGTAGCCTCAG TCTTCCCATCAGGCTCTCAGCTCAGCCTGAGTGTTGAGGCCCCAGTGGCTGCTCTGG GGGCCTCCTGAGTTTCTCATCTGTGCCCCTCCCTCCCTGGCCCAGGTGAAGGTGTGGT TCCAGAACCGGAGGACAAAGTACAAACGGCAGAAGCTGGAGGAGGAAGGGCCTGA GTCCGAGCAGAAGAAGAAGGGCTCCCATCACATCAACCGGTGGCGCATTGCCACGA AGCAGGCCAATGGGGAGGACATCGATGTCACCTCCAATGACTAGGGTGGGCAACCAC AAACCCACGAGGGCAGAGTGCTGCTTGCTGCTGGCCAGGCCCCTGCGTGGGCCCAA GCTGGACTCTGGCCACTCCCTGGCCAGGCTTTGGGGAGGCCTGGAGTCATGGCCCCA CAGGGCTTGAAGCCCGGGGCCGCCATTGACAGAGGGACAAGCAATGGGCTGGCTGA GGCCTGGGACCACTTGGCCTTCTCCTCGGAGAGCCTGCCTGCCTGGGCGGGCCCGCC CGCCACCGCAGCCTCCCAGCTGCTCTCCGTGTCTCCAATCTCCCTTTTGTTTTGATGCA TTTCTGTTTTAATTTATTTTCCAGGCACCACTGTAGTTTAGTGATCCCCAGTGTCCCCC TTCCCTATGG (SEQ ID NO: 131), the two designed guide RNA sequences (E-crRNA1 and E-crRNA2) are: AAUUUCUACUAUUGUAGAUUUGGUUGCCCACCCUAGUCAU (SEQ ID NO: 132); AAUUUCUACUAUUGUAGAUUUACUUUGUCCUCCGGUUCUG (SEQ ID NO: 133) (the underlined area is the target area).

When the fusion degree of HEK 293T cells reached 70/6-80%, we plated them in a 12-well plate at a density of 8×10⁴ cells per well. Six to eight hours after plating, transfection was performed by adding, in sequence, 1 μg of either Gs12-10 or enAsCas12a eukaryotic expression vector, 1 μg of single or tandem guide RNA expression vectors, and 10 μL of Jetprime reagent to 200 μL of Jetprime Buffer. The mixture was then pipetted and incubated at room temperature for 10 minutes. The incubated mixture was added to the culture medium covered with cells for transfection. After incubating at 37° C. for 72 hours, the culture medium was discarded and 100 μL of PBS was used to perform cell resuspension to extract the genome of cells. PCR amplification was performed on the target site of transfected positive cells to edit the nearby sequences. The changes in target bands were observed by T7EN1 digestion and agarose gel electrophoresis. The negative control template consisted of the genomic DNA from untransfected HEK293T cells in normal culture.

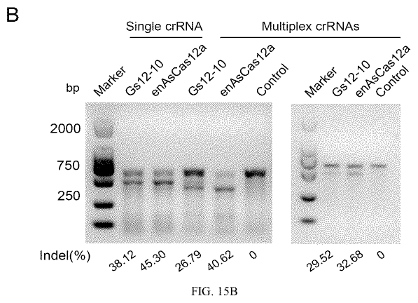

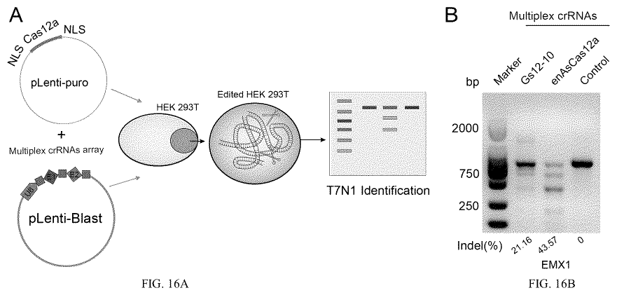

As shown in FIGS. 15A-16B, the CRISPR-Gs12-10 system exhibits high editing activity for both single genes, multiple genes, and multiple loci of a single gene simultaneously. In genome editing of a single RUNX1 gene at a single locus, the editing efficiency of Gs12-10 and the known enhanced enAsCas12a are 38.12% and 45.30%, respectively, with similar activity (FIGS. 15A-15B). When genome editing is performed on both RUNX1 and FANCF simultaneously, compared to editing a single RUNX1 gene, the editing efficiency of Gs12-10 and the known enhanced enAsCas12a are 26.79% and 40.62%, respectively. Although their activity decreases, the activity of Gs12-10 is much lower than that of the enAsCas12a protein, and further research is needed. However, the genome editing on FANCF gene also exhibits high editing activity, at 29.52% and 32.68%, respectively. In FIGS. 16A-16B, when editing simultaneously for two loci of the same EMX1 gene, the editing activity of Gs12-10 and the known enhanced enAsCas12a remains at 21.16% and 43.57%, respectively. Next, some mutations can be used to modify the Gs12-10 protein and improve its genome editing activity. This suggests that the newly identified Gs12-10 protein possesses genome editing capabilities, indicating its suitability for RNA-guided genome engineering.

The above are only preferred embodiments of the present invention, and are not intended to limit the present invention in any form. Although the present invention has been disclosed in the above-preferred embodiments, they are not intended to limit the present invention. Any technician who is familiar with the present invention can make a slight change or modification into the equivalent embodiments of equivalent changes by using the technical content of the above-mentioned hints without departing from the scope of the technical solution of the present invention. But as long as the technical content is not deviated from the technical solution of the present invention, any simple modifications, equivalent changes and modifications made to the above embodiments according to the technical substance of the present invention are still within the scope of the present invention.

What is claimed is:

1. An endonuclease in a clustered regularly interspaced short palindromic repeats (CRISPR)/CRISPR associated (Cas) system, wherein the endonuclease is a Gs12-10 protein with the amino acid sequence as shown in SEQ ID NO: 1.

2. A fusion protein, comprising the endonuclease of claim 1 and other modified parts.

3. A polynucleotide, wherein the polynucleotide encodes the endonuclease of claim 1, or a fusion protein comprising the endonuclease and other modified parts.

4. A vector, wherein the vector comprises the polynucleotide of claim 3.

5. A host cell, wherein the host cell comprises the polynucleotide of claim 3 or a vector comprising the polynucleotide.

6. A method of gene editing, comprising using the endonuclease of claim 1, a fusion protein comprising the endonuclease and other modified parts, a polynucleotide encoding the endonuclease or the fusion protein, a vector comprising the polynucleotide, or a host cell comprising the polynucleotide or the vector.

7. The method of claim 6, wherein the gene editing comprises modifying genes, knocking out the genes, altering an expression of gene products, repairing mutations, or inserting polynucleotides in prokaryotic genomes, eukaryotic genomes, or in vitro genes.

8. A CRISPR/Cas gene editing system, comprising the endonuclease of claim 1, a fusion protein comprising the endonuclease and other modified parts, a polynucleotide encoding the endonuclease or the fusion protein, a vector comprising the polynucleotide, or a host cell comprising the polynucleotide or the vector.

9. The CRISPR/Cas gene editing system of claim 8, further comprising a direct repeat sequence capable of binding to the endonuclease and a guiding sequence capable of targeting a target sequence.

10. A visual nucleic acid detection kit, comprising the endonuclease of claim 1, a single-stranded DNA fluorescence quenching reporter, and a guide RNA paired with a target nucleic acid.
